(19) United States
(12) Patent Application Publication
(10) Pub. No.: US 2025/0189529 A1
(43) Pub. Date: Jun. 12, 2025

(54) HIGH RISK BIOMARKERS FOR MYELOMA PRECURSOR DISEASE PROGRESSION AND METHODS OF USE THEREOF

(71) Applicants: Dana-Farber Cancer Institute, Inc., Boston, MA (US); The Broad Institute, Inc., Cambridge, MA (US); The General Hospital Corporation, Boston, MA (US)

(72) Inventors: Elizabeth Lightbody, Boston, MA (US); Irene M. Ghobrial, Boston, MA (US); Steven Carr, Cambridge, MA (US); Denkanikota R. Mani, Cambridge, MA (US); Michael Gillette, Boston, MA (US)

(73) Assignees: Dana-Farber Cancer Institute, Inc., Boston, MA (US); The Broad Institute, Inc., Cambridge, MA (US); The General Hospital Corporation, Boston, MA (US)

(21) Appl. No.: 18/972,433

(22) Filed: Dec. 6, 2024

Related U.S. Application Data
(60) Provisional application No. 63/607,991, filed on Dec. 8, 2023.

(51) Int. Cl. G01N 33/574; G01N 33/68

(52) U.S. Cl. CPC G01N 33/57488; G01N 33/57492; G01N 33/6863; G01N 33/6872

(57) ABSTRACT

Provided herein are biomarkers and combinations of biomarkers for use in manufacturing panels for determining whether a patient is at risk for multiple myeloma precursor disease progression. Also provided herein are methods of treatment for subjects identified as being at risk for multiple myeloma precursor disease progression.

### CROSS-REFERENCE TO RELATED APPLICATION

This application claims priority to and benefit of U.S. Provisional Application No. 63/607,991, filed Dec. 8, 2023, the entire contents of which are incorporated herein by this reference.

### BACKGROUND OF THE INVENTION

Multiple myeloma (MM) is a cancer that develops in human plasma cells. Plasma cells form a part of the immune system, in particular by making antibodies called immunoglobulins that bind to antigens. In multiple myeloma, the plasma cells may produce an abnormal protein known as monoclonal immunoglobulin, also called “M-spike.”

MM is typically preceded by an MM precursor disease. A patient at risk for MM first develops a medical condition known as monoclonal gammopathy of undetermined significance (MGUS). Following MGUS, the patient may develop smoldering multiple myeloma (SMM).

MM precursor diseases, such as MGUS or SMM, must be monitored for progression into MM in order to provide the best treatment outcomes in patients. Further, patients having MM precursor diseases may remain stable, or may unknowingly rapidly progress into MM. However, bone marrow (BM) biopsies are an invasive and painful procedure. Therefore, repeat BM sampling in patients with MM precursor diseases for continuous burden monitoring is not feasible. Accordingly, less invasive and painful methods for monitoring MM precursor diseases in patients are urgently needed.

### SUMMARY OF THE INVENTION

As described below, the present disclosure features compositions and methods for characterizing multiple myeloma precursor disease in a subject, and selecting subjects for treatment to disrupt the subject's progression to multiple myeloma (MM). Also featured herein are methods of treatment of MM in a subject or treatment of a subject having a MM precursor disease.

In an aspect, the present disclosure provides a panel. The panel includes capture reagents that specifically bind two or more polypeptide markers, where the polypeptide markers are: B-cell maturation antigen (BCMA), cochlin (COCH), contactin 5 (CNTN5), intercellular adhesion molecule 3 (ICAM3), and/or TNF receptor superfamily member 13b (TNFRSF13B), or polynucleotides encoding said markers.

In another aspect, the present disclosure provides a method of treating a selected subject having a multiple myeloma precursor disease. The method involves: administering an effective amount of an agent for treating a multiple myeloma precursor disease to a selected subject, where the subject is selected by detecting an increase in a polypeptide marker, where the polypeptide marker is COCH, ICAM3, TNFRSF13B, and/or CNTN5, or in a polynucleotide encoding said marker in a biological sample of a subject relative to a reference.

In another aspect, the present disclosure provides a method for characterizing multiple myeloma precursor disease in a subject. The method involves detecting one or more polypeptide markers, where the markers are selected from Table 1, Table 2, Table 3, and/or Table 4, or a polynucleotide encoding said marker in a biological sample from the subject, thereby characterizing the disease in the subject, where the polypeptide marker is not B-cell maturation antigen (TNFRSF17/BCMA).

In another aspect, the present disclosure provides a method for monitoring multiple myeloma precursor disease progression in a subject. The method involves: a) detecting a polypeptide marker, where the polypeptide marker is cochlin (COCH), contactin 5 (CNTN5), intercellular adhesion molecule 3 (ICAM3), and/or TNF receptor superfamily member 13b (TNFRSF13B) or a polynucleotide encoding said marker in two or more biological samples from the subject, where the first sample is collected at an earlier point in time than the second sample; and b) comparing a level of the markers, where an increase is indicative that the multiple myeloma precursor disease has progressed in the subject, and failure to detect an increase is indicative that the multiple myeloma precursor disease has not progressed.

In another aspect, the present disclosure provides a method for assessing a subject's risk of multiple myeloma precursor disease progression. The method involves detecting a polypeptide marker, where the polypeptide marker is cochlin (COCH), contactin 5 (CNTN5), intercellular adhesion molecule 3 (ICAM3), and/or TNF receptor superfamily member 13b (TNFRSF13B) or a polynucleotide encoding said marker in a biological sample from the subject, thereby assessing the risk of multiple myeloma precursor disease progression in the subject.

In another aspect, the present disclosure provides a kit including: (a) a panel of markers of any of the above aspects, or embodiments thereof; and (b) instructions for using the panel in the methods of any of the above aspects, or embodiments thereof.

In any of the above aspects, or embodiments thereof, the panel further includes markers bound to the capture reagents.

In any of the above aspects, or embodiments thereof, the capture reagent is fixed to a solid substrate. In any of the above aspects, or embodiments thereof, the substrate is a plate, chip, bead, microfluidic platform, membrane, planar microarray, or suspension array

In any of the above aspects, or embodiments thereof, the polypeptide capture reagent is an antibody or antigen binding fragment thereof. In any of the above aspects, or embodiments thereof, the polynucleotide capture reagent is an aptamer, or a probe.

In any of the above aspects, or embodiments thereof, the detecting is by immunoassay. In any of the above aspects, or embodiments thereof, the detecting is by measuring hybridization to a detectable probe or aptamer.

In any of the above aspects, or embodiments thereof, the agent includes one or more of an immunomodulatory agent, a proteasome inhibitor, a corticosteroid, and a CD38 monoclonal antibody.

In any of the above aspects, or embodiments thereof, the polypeptide marker is: cochlin (COCH), contactin 5 (CNTN5), intercellular adhesion molecule 3 (ICAM3), and/or TNF receptor superfamily member 13b (TNFRSF13B).

In any of the above aspects, or embodiments thereof, the method characterizes the disease as low-risk smoldering multiple myeloma (SMM), or high-risk SMM.

In any of the above aspects, or embodiments thereof, the polypeptide marker is: TNF receptor superfamily member 13b (TNFRSF13B), contactin 5 (CNTN5), Fc receptor-like protein 5 (FCRL5), TNF receptor superfamily member 13 (TNFSF13), intercellular adhesion molecule 3 (ICAM3), CD5 antigen-like (CD5L), SLAM family member 7 (SLAMF7), cluster of differentiation 79B (CD79B), marginal zone B and B1 cell specific protein (MZB1), interleukin receptor 5 subunit alpha (IL5RA), and/or neuronal-specific septin-3 (SEPTIN3).

In any of the above aspects, or embodiments thereof, the subject is undergoing treatment for multiple myeloma precursor disease.

In any of the above aspects, or embodiments thereof, the biological sample is one or more of: blood, serum, plasma, or bone marrow biopsy. In any of the above aspects, or embodiments thereof, the sample is plasma.

In any of the above aspects, or embodiments thereof, the multiple myeloma precursor disease is monoclonal gammopathy of undetermined significance (MGUS) or smoldering multiple myeloma (SMM).

In any of the above aspects, or embodiments thereof, the assessing the subject's risk of multiple myeloma precursor disease progression involves assessing the risk that the multiple myeloma precursor disease will, within a timeframe, progress into multiple myeloma. In any of the above aspects, or embodiments thereof, the assessing the subject's risk of multiple myeloma precursor disease progression involves identifying the multiple myeloma precursor disease as a high-risk multiple myeloma precursor disease.

In any of the above aspects, or embodiments thereof, the method further involves detecting B-cell maturation antigen (TNFRSF17/BCMA), or a polynucleotide encoding BCMA in a biological sample from the subject.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, nucleic acid molecule, polypeptide, or fragments thereof. In some embodiments, the agent is an effective for treating multiple myeloma or multiple myeloma precursor disease.

By “alteration” is meant a change in the structure, expression levels or activity of a marker or clinical variable as detected by standard art known methods such as those described herein. The alteration can be an increase or a decrease. As used herein, an alteration includes a 10%, 20%, 30%, 40%, 50%, 60%, 70%, 80%, 90% or greater change in the level of the marker or clinical variable.

By “ameliorate” is meant decrease, suppress, attenuate, diminish, arrest, or stabilize the development or progression of a disease. In some embodiments, the diseases include multiple myeloma (MM), such as newly diagnosed multiple myeloma (NDMM), or a symptom thereof, and MM precursor diseases, such as smoldering multiple myeloma (SMM) and monoclonal gammopathy of undetermined significance (MGUS), or a symptom thereof.

By “biological sample” is meant a sample obtained from a subject. In some embodiments, a biological sample is a blood, sera, plasma, or bone marrow sample.

By “capture molecule” is meant an agent that specifically binds to an analyte. Capture molecules are used in, for example, ligand binding assays, immune assays, and protein or nucleic acid molecule capture assays. In one embodiment, a biological sample is characterized using a capture array, in which reagents, which are usually antibodies, but can also be alternative protein scaffolds, peptides or nucleic acid aptamers, are used to detect target molecules in mixtures, such as biological samples (e.g., blood, plasma or tissue extracts). In diagnostics, capture arrays are used to carry out multiple immunoassays in parallel, both testing for several analytes in individual blood/sera for example and testing many blood/serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, optionally in a multiplex array format against multiple protein targets.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

“Detect” refers to identifying the presence, absence or amount of an analyte to be detected. In embodiments, the analyte is a polypeptide or polynucleotide marker (e.g., B-cell maturation antigen (BCMA), cochlin (COCH), contactin 5 (CNTN5), intercellular adhesion molecule 3 (ICAM3), and TNF receptor superfamily member 13b (TNFRSF13B) polypeptides, or polynucleotides encoding said polypeptides).

By “disease” is meant any condition or disorder that damages or interferes with the normal function of a cell, tissue, or organ. Examples of diseases include multiple myeloma (MM), such as newly diagnosed multiple myeloma (NDMM), and MM precursor diseases, such as smoldering multiple myeloma (SMM) and monoclonal gammopathy of undetermined significance (MGUS).

By “effective amount” is meant the amount of an agent required to ameliorate the symptoms of a disease of a patient relative to an untreated patient having the disease. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In an embodiment, an effective amount is the amount required to reduce a rate of progression of a MM precursor disease. In an embodiment, an effective amount is the amount required to ameliorate MM, NDMM, or a MM precursor disease, such as MGUS or SMM.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

By “increase” is meant to alter positively relative to a reference. An increase may be by 1%, 5%, 10%, 25%, 30%, 50%, 75%, 100%, or more, or by 1.5-fold,-fold 2-fold, 3-fold, 4-fold, 5-fold, 6-fold, 7-fold, 8-fold, 9-fold, 10-fold, 25-fold, 50-fold, 75-fold, 100-fold, or more.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, and or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” or “biomarker” is meant any analyte, including protein or polynucleotides having an alteration in expression, level, or activity that is associated with a disease, disorder, or state. In some embodiments, the marker is a polypeptide, polynucleotide or other analyte whose level is detected during the characterization of a disease (e.g., MM precursor disease or MM). In some embodiments, an alteration in a marker's expression level, concentration, abundance, activity or structure is detected. In some embodiments, an alteration in a marker is detected over time (e.g., over days, weeks, months, years). In some embodiments, the marker is COCH, ICAM3, TNFRSF13B, CNTN5, or a combination thereof. In some embodiments, the marker may further include BCMA.

By “multiple myeloma precursor disease” is meant a pathological condition indicating that a subject has the propensity to develop multiple myeloma. Exemplary multiple myeloma precursor diseases include SMM and MGUS.

By “multiple myeloma precursor disease progression” is meant the progression in a subject of a multiple myeloma precursor disease from a less advanced stage to a more advanced stage. For example, the progression of a subject from MGUS to SMM, or from a multiple myeloma precursor disease to multiple myeloma.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing, a disorder or condition.

By “reduce” is meant to alter negatively relative to a reference. A reduction may be by 1%, 5%, 10%, 25%, 30%, 50%, 75%, 100%, or more, or by 1.5-fold,-fold 2-fold, 3-fold, 4-fold, 5-fold, 6-fold, 7-fold, 8-fold, 9-fold, 10-fold, 25-fold, 50-fold, 75-fold, 100-fold, or more.

By “reference” is meant a standard or control condition. In one embodiment, the level of a marker in a biological sample obtained from a subject having a multiple myeloma precursor disease is compared to the level of that marker in a healthy control subject or is compared to the level of that marker in a subject having multiple myeloma. In some embodiments, the reference is a healthy reference range.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, about less than 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., of at least about 42° C., or of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated. In embodiments, a treatment involves one or more of characterizing a biological sample and/or a neoplasia, such as MM, NDMM, or a MM precursor disease, such as MGUS or SMM, monitoring a patient and/or neoplasia, such as MM, NDMM, or a MM precursor disease, such as MGUS or SMM, in the patient, and prognosis.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art. In some cases, a range of normal tolerance in the art is within 1 or 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides a schematic showing a description of the subjects used in the study cohort of Example 1, wherein the subjects present in the cohort are characterized as follows: healthy; having monoclonal gammopathy of undetermined significance (MGUS); having smoldering multiple myeloma (SMM); or as having multiple myeloma (MM). As shown in the schematic, peripheral blood samples were taken from each subject in the study cohort and used in analysis.

FIG. 2 provides a graph showing the study cohort for Example 1. Non-progressors and progressors (i.e., subjects that advanced from a less advanced MM disease stage to a more advanced disease stage, such as from SMM to newly diagnosed multiple myeloma (NDMM)) were time matched for clinical follow-up time. As shown in the graph, a subset of progressors had sequential samples collected at both MM precursor and active MM timepoints, while the remaining patients had samples banked at only the precursor stage timepoint. The graph indicates that precursor samples ranged between 1.03-5.88 years prior to progression of active MM, with a median of 1.74 years. The median clinical follow-up time was 7.05 years.

FIGS. 3A-3B provides bar graphs showing the identification of precursor and myeloma-specific dysregulated proteins. Between 2500-3000 proteins were identified across all patients. Of this set, a number of proteins were significantly dysregulated in precursor and active MM patients compared to healthy individuals (Healthy vs. MGUS=222; vs. SMM=423; vs. NDMM=494). FIG. 3A provides a more detailed breakdown of the subsets of dysregulated proteins for each condition. In FIG. 3B, the bars are ordered in the following order, from bottom to top: significant upregulation; significant downregulation; and no significant change.

FIGS. 4A-4D provide graphs showing that Proximity Extension Assay (PEA) technology was able to capture well-defined proteomic alterations in MM disease progression. FIG. 4A shows that the Normalized Protein expression (NPX) levels for TNFRSF17/BCMA change with MM disease progression. FIG. 4B shows that the expression levels for SLAMF7 change with MM disease progression. FIG. 4C shows that the expression levels for SDC1/CD138 change with MM disease progression. FIG. 4D shows that the expression levels for IL-6 change with MM disease progression. For each graph, the bars above the graphs provide p-values, and the groups are, from left to right: expression in healthy subjects; expression in MGUS subjects; expression in SMM subjects; and expression in NDMM subjects.

FIG. 5 provides box plots showing that an analysis of the MM plasma proteome revealed disease-specific signals. The box-plots represent exemplary proteins shown to be upregulated in MM, as compared both to healthy subjects and to other forms of cancer and/or neoplasia.

FIGS. 6A-6E provide graphs showing particular markers which were shown to be predictive of MM disease progression from SMM. In these graphs, non-progressor expression is compared to progressor expression.

FIGS. 7A-7C provide volcano plots and graphs showing the differential expression and Gene Set Pathway Enrichment Analyses of Healthy vs. MGUS, SMM and NDMM. Many proteins were discovered that were significantly differentially expressed in (FIG. 7A) MGUS, (FIG. 7B) SMM, and (FIG. 7C) NDMM patients vs. healthy donors (n=222, 423 and 494). DE proteins with an FDR <0.01 and absolute fold change >1.5 were selected for display in the volcano plots. GSEA analysis highlighted the top 12 pathways dysregulated for each disease stage comparison. In each volcano plot, proteins to the left of the left vertical bar were downregulated, proteins to the right of the right vertical bar were upregulated, and proteins between the two vertical bars showed no significance. In FIG. 7A, right panel, the calcium signaling pathway, the cytokine-cytokine receptor interaction pathway, and the JAX-STAT signaling pathway were upregulated, and the remainder of the pathways were downregulated. In FIG. 7B, right panel, the ERBB signaling pathway and the MAPK signaling pathway were upregulated, and the remainder were downregulated. In FIG. 7C, right panel, the ERBB signaling pathway, the FcγR mediated phagocytosis pathway, the T cell receptor signaling pathway, and the regulation of actin cytoskeleton pathway were upregulated, and the remainder were downregulated.

FIG. 8 provides box plots showing that BCMA and Transmembrane Activator and CAML Interactor (TACI) protein plasma levels are not only elevated at baseline in progressors vs. non-progressors but show significant dynamic increases in expression in sequential samples from SMM-MM progressors. Linear mixed effect model was implemented using Empirical Bayes approach. Model consisted of the Assigned Disease Group and the interaction of Timepoint and Assigned Disease Group as the independent covariates. Same patient samples were accounted for using a random effect. After model fitting, contrasts of interest were evaluated to identify proteins that were significantly differential following multiple testing correction using the Benjamini-Hochberg approach. BCMA and TACI levels significantly increased over time to progression within 17 SMM-MM progressors with sequential samples.

FIG. 9 provides a heat map showing the results of scRNA-seq based cellular mapping of signals detected in plasma. Patients with plasma proteomics also had scRNA-seq performed on their paired bone marrow samples collected at the same time point as their blood samples. The expression of the 5 proteins associated with progression within the plasma proteome data was assessed in tumor and immune cells from the bone marrow to better understand what cell types were contributing to their signals in the blood. TNFRSF17, TNFRSF13B, ICAM3 and CNTN5 were all expressed higher in malignant vs. non-malignant plasma cells, suggesting that these were proteins in the blood providing a nice readout of malignant plasma cell burden. Additionally, TNFRSF13B, ICAM3 and COCH were expressed by other immune cell types which suggested immune cell dysregulation was ongoing and may also be measured using peripheral blood samples.

### DETAILED DESCRIPTION OF THE INVENTION

The present disclosure features compositions and methods for characterizing multiple myeloma precursor disease in a subject, and selecting subjects for treatment to disrupt the subject's progression to multiple myeloma (MM). Also featured herein are methods of treatment of MM in a subject or treatment of a subject having a MM precursor disease.

The invention is based, at least in part, on the discovery of polypeptide and polynucleotide biomarkers (e.g., B-cell maturation antigen (BCMA), cochlin (COCH), contactin 5 (CNTN5), intercellular adhesion molecule 3 (ICAM3), and TNF receptor superfamily member 13b (TNFRSF13B)) useful for characterizing multiple myeloma precursor disease. In particular, the biomarkers cochlin (COCH), intercellular adhesion molecule 3 (ICAM3), TNF receptor superfamily member 13b (TNFRSF13B), contactin 5 (CNTN5), and the biomarker B-cell maturation antigen (TNFRSF17/BCMA), were shown to be predictive of MM precursor disease progression. Accordingly, the invention features, for example, the use of capture molecules that bind a COCH, ICAM3, TNFRSF13B, CNTN5 polypeptide or a polynucleotide encoding said polypeptide, alone or in combination with B-cell maturation antigen (BCMA), as well as combinations thereof, to characterize a MM precursor disease, and/or to select a subject for treatment.

### Hematological Malignancies

Multiple myeloma (MM) is a plasma cell dyscrasia. Plasma cell dyscrasias are cancers of the plasma cells. They are produced as a result of malignant proliferation of a monoclonal population of plasma cells that may or may not secrete detectable levels of a monoclonal immunoglobulin or paraprotein commonly referred to as M protein. Further non-limiting examples of plasma cell dyscrasias include monoclonal gammopathy of undetermined significance (MGUS), smoldering multiple myeloma (SMM), symptomatic multiple myeloma, Waldenstrom macroglobulinemia (WM), amyloidosis (AL), plasmacytoma syndrome (e.g., solitary plasmacytoma of bone, extramedullary plasmacytoma), light chain deposition disease, and heavy-chain disease. MGUS, SMM, and symptomatic MM represent a spectrum of the same disease.

MGUS is characterized by a serum monoclonal protein (<30 g/L), <10% plasma cells in the bone marrow, and absence of end-organ damage. Asymptomatic MGUS stage consistently precedes multiple myeloma (MM). MGUS is present in 3% of persons >50 years and in 5%>70 years of age. The average risk of progression to MM or a related disorder is 1% per year. Patients with risk factors consisting of an abnormal serum free light chain ratio, non-immunoglobulin G (IgG) MGUS, and an elevated serum M protein (≥15 g/1) have a risk of progression at 20 years of 58%, compared with 37% among patients with two risk factors, 21% for those with one risk factor, and 5% for individuals with no risk factors. The cumulative probability of progression to active MM or amyloidosis is 51% at 5 years, 66% at 10 years and 73% at 15 years; the median time to progression was 4.8 years

Smoldering Multiple Myeloma (SMM) also known as asymptomatic MM is characterized by having a serum immunoglobulin (Ig) G or IgA monoclonal protein of 30 g/L or higher and/or 10% or more plasma cells in the bone marrow but no evidence of end-organ damage.

Symptomatic or Active Multiple Myeloma (MM) is a form of cancer that affects a type of white blood cell called the plasma cell. Multiple myeloma appears in the bone marrow, which is the soft tissue inside the bones that makes stem and immune cells. In multiple myeloma, plasma cells, which mature from stem cells and typically produce antibodies to fight germs and other harmful substances, become abnormal. These abnormal cells are called myeloma cells. In 2021, an estimated 34,920 cases of multiple myeloma were diagnosed in the United States and over 12,410 patient deaths associated with multiple myeloma were reported. As the most common type of plasma cell cancer, effective treatment requires an accurate diagnosis and precise treatment.

Symptomatic or active MM is characterized by any level of monoclonal protein and the presence of end-organ damage that consists of the SLIM-CRAB criteria (bone marrow plasma cell percentage ≥60%, involved: uninvolved free light chain ratio >100, >1 focal lesion on MRI, hypercalcemia, renal insufficiency, anemia, or bone lesions). MM is a plasma cell malignancy that characteristically involves extensive infiltration of bone marrow (BM), with the formation of plasmacytomas, as clusters of malignant plasma cells inside or outside of the BM milieu. Consequences of this disease are numerous and involve multiple organ systems. Disruption of BM and normal plasma cell function leads to anemia, leukopenia, hypogammaglobulinemia, and thrombocytopenia, which variously result in fatigue, increased susceptibility to infection, and, less commonly, increased tendency to bleed. Disease involvement in bone creates osteolytic lesions, produces bone pain, and may be associated with hypercalcemia.

### Conventional Detection Methods

To date, the gold standard for characterizing MM disease state has involved a bone marrow biopsy. In embodiments, the present disclosure provides less invasive methods for characterizing the disease state of a patient in a biological sample, such as a blood, plasma, or serum sample. The methods of the invention are suitable for use alone, or if desired, may be used in concert with one or more of the following conventional diagnostic methods.

The criteria for the diagnosis of MM, SMM, and MGUS are detailed in Table 1 below. Distinction among these disease states informs treatment decisions and prognostic recommendations.

### Table 1. Conventional Criteria for the Diagnosis of MM, SMM, and MGUS

TABLE 1
Conventional criteria for the diagnosis of MM, SMM, and MGUS

| Disorder | Disease definition |
|---|---|
| MGUS | Serum monoclonal protein level <3 g/dL, bone marrow plasma cells |
| | 10%, and absence of end-organ damage, such as lytic bone lesions, |
| | anemia, hypercalcemia, or renal failure, that can be attributed to a |
| | plasma cell proliferative disorder. |
| SMM | Serum monoclonal protein (IgG or IgA) level ≥3 g/dL and/or bone |
| | marrow plasma cells l ≥10%, absence of end-organ damage, such as |
| | lytic bone lesions, anemia, hypercalcemia, or renal failure that can be |
| | attributed to a plasma cell proliferative disorder. |
| | Alternatively, or additionally, SMM may be defined by the absence of |
| | markers (e.g., Myeloma Defining Event markers), by bone-marrow |
| | plasma cell infiltration ε60%, by serum-free light chain ratio ε100, |
| | and/or by >1 focal lesion in the skeleton on magnetic resonance |
| | imaging analysis (see, Rajkumar SV, et al. International Myeloma |
| | Working Group updated criteria for the diagnosis of multiple |
| | myeloma. Lancet Oncol 15, e538-548 (2014)). In some instances, |
| | SMM may be associated with organ damage. |
| MM | Bone marrow plasma cells ≥10%, presence of serum and/or urinary |
| | monoclonal protein (except in patients with true nonsecretory multiple |
| | myeloma), plus evidence of lytic bone lesions, anemia, |
| | hypercalcemia, or renal failure that can be attributed to the underlying |
| | plasma cell proliferative disorder. |

Approximately half of patients with SMM will progress within the first 5 years from diagnosis, yet not all patients with SMM progress. Therefore, patients with SMM are typically observed until end-organ damage (e.g., kidney failure, bone disease, anemia) occurs, which signifies progression to overt MM, warranting treatment.

While multiple myeloma (MM) is always preceded by two precursor conditions, clinicians and patients have long struggled to identify the chances that a patient's condition will progress from one precursor condition into the next or into MM. Only a fraction of patients with MGUS will ever have their condition progress to SMM, and only a fraction of patients with SMM will have their condition progress into MM. A chief difficulty for a patient with a precursor condition is thus the uncertainty of the patient's prospects of their condition developing into a rare and serious cancer. The uncertainty of whether MM will develop is compounded by uncertainty of whether and when MGUS could develop into SMM, or whether and when SMM could develop into MM. The ambiguity surrounding the patient's future condition and timeline for that condition creates stress for the patient and their family and friends and creates difficulties for clinicians in managing the patient's care and the patient's expectations.

Given the severe disadvantages associated with the lack of predictability for the condition of a patient with an MM precursor condition, researchers and clinicians have long investigated MM precursor conditions and sought signs and signals that an MM precursor condition could progress or a timeline on which it will progress. But while countless hours/years and resources have been invested in this research and multiple techniques have been developed, a reliable manner of estimating a patient's risk has not been identified. The current best-available technique, while an improvement on previous approaches, still fares little better than a coin flip (i.e., a C-statistic of 0.530) in terms of predictive reliability for disease progression. Accordingly, the methods of the present disclosure involve improved detection of the progression of MM from MM precursor conditions and/or involve detection of such progression using less invasive methods than bone marrow biopsy.

### Detection of Markers

The present disclosure provides for the detection of biomarkers and combinations of biomarkers. In embodiments, the biomarker is one or more of COCH, ICAM3, TNFRSF13B, CNTN5. In embodiments, the combination of biomarkers comprises two or more of COCH, ICAM3, TNFRSF13B, and CNTN5. In embodiments, the combination of biomarkers also comprises TNFRSF17/BCMA. In some embodiments, a level of marker is detected at a single time point or serial values are annotated over time (e.g., days, weeks, months). In some embodiments, levels of a marker are detected at 3, 4, 5, 6, 7, 8, 9, 10, 11, or 12-month time intervals from the date of MGUS or SMM diagnosis. In some embodiments, levels of a marker described herein are detected prior to, during or following initiation of precursor treatment. In some embodiments, levels of a marker are detected in a biological sample (e.g., blood, serum, plasma, or bone marrow biopsy). In embodiments, the present disclosure provides for the identification of a high-risk version of an MM precursor disease. High risk MM precursor disease includes, for example, high-risk MGUS, or high-risk SMM. Identification of high-risk MM precursor disease indicates that the precursor disease is more likely to progress to a more advanced stage of MM precursor disease (e.g., from MGUS to SMM) or is more likely to progress to active MM or NDMM.

In some instances, the level of the marker is increased relative to a reference level of the marker if the level of the marker is at least 1.5-fold, at least 2-fold, at least 3-fold, at least 4-fold, at least 5-fold, at least 6-fold, at least 7-fold, at least 8-fold, at least 9-fold, at least 10-fold, at least 20-fold, at least 25-fold, at least 50-fold, at least 75-fold, or at least 100-fold higher, or at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 1,500%, at least 2,000%, at least 2,500%, at least 3,000%, at least 3,500%, at least 4,000%, at least 4,500%, or at least 5,000% higher than the control level of the marker. In some instances, the level of the marker is decreased relative to a reference level of the marker if the level of the marker is at least 1.5-fold, at least 2-fold, at least 3-fold, at least 4-fold, at least 5-fold, at least 6-fold, at least 7-fold, at least 8-fold, at least 9-fold, at least 10-fold, at least 20-fold, at least 25-fold, at least 50-fold, at least 75-fold, or at least 100-fold lower, or at least 10%, at least 20%, at least 30%, at least 40%, at least 50%, at least 60%, at least 70%, at least 80%, at least 90%, at least 95%, at least 97%, or 100% lower than the control level of the marker. In some instances, the reference level of the marker is the level of the marker in a corresponding sample (e.g., same tissue type as the test sample) from a healthy human subject (e.g., a human subject of similar age who does not have SMM, MGUS, or MM).

In some instances, the reference level of the marker is the median level of the marker in a panel of corresponding samples (e.g., same tissue type as the test sample) for one or more (e.g., 1, 2, 3, 4, 5, 6, 7, 8, 9, 10, 15, 20, 25, 30, 35, or 40 or more) healthy human subjects (e.g., human subjects of similar age who do not have SMM or MM) and/or for one or more (e.g., 1, 2, 3, 4, 5, 6, 7, 8, 9, 10, 15, 20, 25, 30, 35, or 40 or more) human subjects with SMM, MGUS, or MM. In some instances, the reference level of the marker is the mean level of the marker in a panel of corresponding samples (e.g., same tissue type as the test sample) for one or more (e.g., 1, 2, 3, 4, 5, 6, 7, 8, 9, 10, 15, 20, 25, 30, 35, or 40 or more) healthy human subjects (e.g., human subjects of similar age who do not have SMM or MM) and/or for one or more (e.g., 1, 2, 3, 4, 5, 6, 7, 8, 9, 10, 15, 20, 25, 30, 35, or 40 or more) human subjects with SMM, MGUS, or MM. In some instances, the reference level of the marker is the first quartile level of the marker in a panel of corresponding samples (e.g., same tissue type as the test sample) for one or more (e.g., 1, 2, 3, 4, 5, 6, 7, 8, 9, 10, 15, 20, 25, 30, 35, or 40 or more) healthy human subjects (e.g., human subjects of similar age who do not have SMM or MM) and/or for one or more (e.g., 1, 2, 3, 4, 5, 6, 7, 8, 9, 10, 15, 20, 25, 30, 35, or 40 or more) human subjects with SMM, MGUS, or MM. In some instances, the reference level of the marker is the third quartile level of the marker in a panel of corresponding samples (e.g., same tissue type as the test sample) for one or more (e.g., 1, 2, 3, 4, 5, 6, 7, 8, 9, 10, 15, 20, 25, 30, 35, or 40 or more) healthy human subjects (e.g., human subjects of similar age who do not have SMM or MM) and/or for one or more (e.g., 1, 2, 3, 4, 5, 6, 7, 8, 9, 10, 15, 20, 25, 30, 35, or 40 or more) human subjects with SMM, MGUS, or MM.

The markers of this invention can be detected by any suitable method. The methods described herein can be used individually or in combination for a more accurate detection of the markers (e.g., biochip in combination with mass spectrometry, immunoassay in combination with mass spectrometry, single cell RNA sequencing, and the like).

One of skill in the art is familiar with how to detect levels of markers in a sample. For example, commercial kits and/or well developed methods are available for detection of creatinine (e.g., the “Creatinine Assay Kit” from Cell Biolabs, Inc.), free light chain ratio (see, e.g., Tosi, et al. Ther Adv Hematol 4:37-41 (2013)), M-spike (see, e.g., Noori, et al. Clinical Chemistry and Laboratory Medicine 59:1963-1971 (2021)), or hemoglobin (e.g., the “Hemoglobin Assay Kit” available from Millipore Sigma) in a sample.

Detection paradigms that can be employed in the invention include, but are not limited to, optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are described below.

### Detection by Immunoassay

In particular embodiments, the markers of the invention are measured by immunoassay. Immunoassay typically utilizes an antibody (or other agent that specifically binds the marker) to detect the presence or level of a marker in a sample. Antibodies can be produced by methods well known in the art, e.g., by immunizing animals with the markers. Markers can be isolated from samples based on their binding characteristics. Alternatively, if the amino acid sequence of a polypeptide marker is known, the polypeptide can be synthesized and used to generate antibodies by methods well known in the art.

This invention contemplates traditional immunoassays including, for example, Western blot, sandwich immunoassays including ELISA and other enzyme immunoassays, fluorescence-based immunoassays, and chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the marker is attached to the surface of an MS probe, such as a pre-activated ProteinChip array. The marker is then specifically captured on the biochip through this reagent, and the captured marker is detected by mass spectrometry.

Detection by Sequencing and/or Probes

In particular embodiments, the markers of the invention are analyzed by a probe-based technique (e.g., RNA-seq).

RNA sequencing (RNA-Seq) is a powerful tool for transcriptome profiling. In embodiments, to mitigate sequence-dependent bias resulting from amplification complications to allow truly digital RNA-Seq, a set of barcode sequences can be used to ensure that every cDNA molecule prepared from an mRNA sample is uniquely labeled by random attachment of barcode sequences to both ends (see, e.g., Shiroguchi K, et al. Proc Natl Acad Sci USA. 2012 Jan. 24; 109 (4): 1347-52). After PCR, paired-end deep sequencing can be applied to read the two barcodes and cDNA sequences. Rather than counting the number of reads, RNA abundance can be measured based on the number of unique barcode sequences observed for a given cDNA sequence. The barcodes may be optimized to be unambiguously identifiable. This method is a representative example of how to quantify a whole transcriptome from a sample.

Detecting a target polynucleotide sequence or fragment thereof associated with a marker that hybridizes to a probe sequence may involve sequencing, FACS, qPCR, RT-PCR, a genotyping array, and/or a NanoString assay (see, e.g., Malkov, et al. “Multiplexed measurements of gene signatures in different analytes using the Nanostring nCounter™ Assay System”, BMC Research Notes, 2: Article No: 80 (2009)), or any of various other techniques known to one of skill in the art. Various detection methods may be used and are described as follows.

Preparation of a library for sequencing may involve an amplification step. Amplification may involve thermocycling or isothermal amplification (such as through the methods RPA or LAMP). Cross-linking may involve overlap-extension PCR or use of ligase to associate multiple amplification products with each other. Amplification can refer to any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a marker.

Detection of the expression level of a marker can be conducted in real time in an amplification assay (e.g., qPCR). In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dyes suitable for this application include, as non-limiting examples, SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

Other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are taught, for example, in U.S. Pat. No. 5,210,015.

Sequencing may be performed on any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91 (13): 5740 (1994), all of which are expressly incorporated by reference).

The sequencing of a polynucleotide can be carried out using any suitable commercially available sequencing technology. In embodiments, the sequencing of a polynucleotide is carried out using a chain termination method of DNA sequencing (e.g., Sanger sequencing). In some embodiments, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLID sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing.

In embodiments, levels of markers in a sample are quantified using targeted sequencing. Methods for targeted sequencing are well known in the art (see, e.g., Rehm, “Disease-targeted sequencing: a cornerstone in the clinic”, Nature Reviews Genetics, 14:295-300 (2013)).

In embodiments, a probe comprises a molecular identifier, such as a fluorescent or chemiluminescent label, a radioactive isotope label, an enzymatic ligand, or the like. The molecular identifier can be a fluorescent label or an enzyme tag, such as digoxigenin, β-galactosidase, urease, alkaline phosphatase or peroxidase, avidin/biotin complex.

Methods used to detect or quantify binding of a probe to a target marker will typically depend upon the molecular identifier. For example, radiolabels may be detected using photographic film or a phosphoimager. Fluorescent markers may be detected and quantified using a photodetector to detect emitted light. Enzymatic labels can be detected by providing the enzyme with a substrate and measuring the reaction product produced by the action of the enzyme on the substrate; and colorimetric labels can be detected by visualizing a colored label.

Specific non-limiting examples of molecular identifiers include radioisotopes, such as 32P, 14C, 1251, 3H, and 131I, fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a molecular identifier, streptavidin bound to an enzyme (e.g., peroxidase) may further be added to facilitate detection of the biotin.

Examples of fluorescent molecular identifiers include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinyl sulfonyl)phenyl]naphthalimide-3,5 disulfonate; N-(4-anilino-1-naphthyl) maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine.

A fluorescent molecular identifier may be a fluorescent protein, such as blue fluorescent protein, cyan fluorescent protein, green fluorescent protein, red fluorescent protein, yellow fluorescent protein or any photoconvertible protein. Colorimetric molecular identifiers, bioluminescent molecular identifiers and/or chemiluminescent molecular identifiers may be used in embodiments of the invention.

Detection of a molecular identifier may involve detecting energy transfer between molecules in a hybridization complex by perturbation analysis, quenching, or electron transport between donor and acceptor molecules, the latter of which may be facilitated by double stranded match hybridization complexes. The fluorescent molecular identifier may be a perylene or a terrylen. In the alternative, the fluorescent molecular identifier may be a fluorescent barcode.

The molecular identifier may be light sensitive, wherein the label is light-activated and/or light cleaves the one or more linkers to release the molecular cargo. The light-activated molecular cargo may be a major light-harvesting complex (LHCII). In another embodiment, the fluorescent molecular label may induce free radical formation.

In an advantageous embodiment, agents may be uniquely labeled in a dynamic manner (see, e.g., international patent application serial no. PCT/US2013/61182 filed Sep. 23, 2012). The unique labels are, at least in part, nucleic acid in nature, and may be generated by sequentially attaching two or more detectable oligonucleotide tags to each other and each unique label may be associated with a separate agent. A detectable oligonucleotide tag may be an oligonucleotide that may be detected by sequencing of its nucleotide sequence and/or by detecting non-nucleic acid detectable moieties to which it may be attached.

In embodiments, the molecular identifier is a microparticle, including, as non-limiting examples, quantum dots (Empodocles, et al., Nature 399:126-130, 1999), or gold nanoparticles (Reichert et al., Anal. Chem. 72:6025-6029, 2000).

### Detection by Biochip

In embodiments, a sample is analyzed by means of a biochip (also known as a microarray). Capture molecules that bind the polypeptides and nucleic acid markers described herein are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In embodiments, a sample is analyzed by means of a protein biochip (also known as a protein microarray). Such biochips are useful in high-throughput low-cost screens to identify alterations in the expression or post-translation modification of a marker, or a fragment thereof. In embodiments, a protein biochip of the invention binds a marker present in a sample and detects an alteration in the level of the marker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); tissue (e.g., bone marrow), a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, CA), Zyomyx (Hayward, CA), Packard BioScience Company (Meriden, CT), Phylos (Lexington, MA), Invitrogen (Carlsbad, CA), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g., RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions include, as non-limiting examples, temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In an embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art.

Detection systems for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In embodiments, the markers of this invention are detected by mass spectrometry (MS). Mass spectrometry is a well-known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1 (2): 880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the invention is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on a collecting membrane, and the array may be analyzed. Subsequently, automated output of the results is provided by using a server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MS″ analysis to be performed. Tandem mass spectrometry allows multiple reactions to be monitored at the same time.

Capillary infusion may be employed to introduce the marker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high-performance liquid chromatography (HPLC) to a mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the invention is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the markers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

A biospecific adsorbent is an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10-8 M.

Protein biochips produced by Ciphergen comprise surfaces having chromatographic or biospecific adsorbents attached thereto at addressable locations. Ciphergen's ProteinChip® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidazole, epoxide) and PG-20 (protein G coupled through acyl-imidazole). Hydrophobic ProteinChip arrays have isopropyl or nonylphenoxy-poly (ethylene glycol) methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidazole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the marker or markers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound markers.

In yet another method, one can capture the markers with a solid-phase bound immuno-adsorbent that has antibodies that bind the markers. After washing the adsorbent to remove unbound material, the markers are eluted from the solid phase and detected by applying to a SELDI biochip that binds the markers and analyzing by SELDI.

The markers bound to the substrates are detected in a gas phase ion spectrometer such as a time-of-flight mass spectrometer. The markers are ionized by an ionization source such as a laser, the generated ions are collected by an ion optic assembly, and then a mass analyzer disperses and analyzes the passing ions. The detector then translates information of the detected ions into mass-to-charge ratios. Detection of a marker typically will involve detection of signal intensity. Thus, both the quantity and mass of the marker can be determined.

### Methods of Selecting Subjects For Treatment

The biomarkers and combinations of biomarkers disclosed herein can be used to select subjects (e.g., humans) with SMM or MGUS that would benefit from early treatment (i.e., before progression to MM). Patients with MM can also present with an increased immune reactivity and, thus, are also more likely to respond to treatment with, e.g., immunotherapy. Thus, prior to undergoing treatment for SMM, MGUS, or MM, subjects (e.g., human) with SMM, MGUS, or MM having one or more markers disclosed herein are selected for treatment, and are predicted to have significantly longer progression-free survival upon treatment (e.g., with immunotherapy) and are, thus, predicted to benefit from treatment (e.g., treatment for MM subjects or early treatment for SMM or MGUS subjects, i.e., treatment before progression from SMM or MGUS to MM). Accordingly, provided herein is a method for identifying a human subject having SMM, MGUS, or MM that would benefit from treatment, the method comprising determining that a sample (e.g., blood, serum, plasma, or bone marrow biopsy) obtained from the human subject has one or more markers disclosed herein, wherein the sample is obtained prior to treatment. In some embodiments, the method comprises obtaining the sample from the human subject. In some embodiments, the human subject has not or is not currently undergoing treatment for SMM, MGUS, or MM at the time the sample is obtained from the human subject. In some embodiments, the human subject has SMM. In some embodiments, the human subject having SMM has high-risk SMM. In some embodiments, the human subject having SMM has high-risk SMM based on the Rajkumar et al., Blood 125:3069-3075 (2015) criteria. In some embodiments, the human subject having SMM has high-risk SMM based on the “20-2-20” criteria. In some embodiments, the human subject having SMM has low- or intermediate-risk SMM based on the “20-2-20” criteria. In some embodiments, the human subject has MGUS. In some embodiments, the human subject has MM. In some embodiments, the human subject has NDMM. In some embodiments, the method further comprises (i.e., after the determining) administering to the human subject a treatment for SMM, MGUS, or MM. In some embodiments in which the human subject has SMM or MGUS, the treatment is administered to the human subject before the SMM or MGUS progresses to MM (e.g., overt MM). In some embodiments, the treatment is a treatment for SMM described herein. In some embodiments, the treatment is a treatment for MGUS described herein. In some embodiments, the treatment is a treatment for MM described herein.

### Treatments

Methods of inhibiting and/or treating cancer and tumors (e.g., a multiple myeloma) in a subject with cancer, a predisposition for developing cancer as identified by methods of the disclosure, or a precursor disease to a cancer (e.g., an MM precursor disease, such as MGUS or SMM), are also contemplated. Methods described herein are useful as clinical or companion diagnostics for therapies or can be used to guide treatment decisions based on clinical response/resistance.

Frontline therapy for MM includes either conventional chemotherapy or high-dose chemotherapy (HDT) supported by autologous or allogeneic stem cell transplantation (SCT), depending on patient characteristics such as performance status, age, availability of a sibling donor, comorbidities, and, in some cases, patient and physician preferences. Other treatments include: bortezomib, thalidomide, lenalidomide, dexamethasone, cyclophosphamide, melphalan, and stem cell transplant. For a patient under 70 years of age, autologous stem cell transplant is proposed after induction.

Non-limiting examples of agents suitable for use to treat a multiple myeloma include a chemotherapeutic agent, radiation, or immunotherapy. Any suitable therapeutic treatment for a particular cancer may be administered. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, tamoxifen, taxol, topotecan hydrochloride, trastuzumab, vinblastine, vincristine and vinorelbine tartrate. Further non-limiting examples of chemotherapeutic agents include an alkylating agent (e.g. busulfan, chlorambucil, cisplatin, cyclophosphamide (Cytoxan), dacarbazine, ifosfamide, mechlorethamine (mustargen), and melphalan), a topoisomerase inhibitor, an antimetabolite (e.g. 5-fluorouracil (5-FU), cytarabine (Ara-C), fludarabine, gemcitabine, and methotrexate), an anthracycline, an antitumor antibiotic (e.g. bleomycin, dactinomycin, daunorubicin, doxorubicin (Adriamycin), and idarubicin), an epipodophyllotoxin, nitrosureas (e.g. carmustine and lomustine), topotecan, irinotecan, doxorubicin, etoposide, mitoxantrone, bleomycin, busultan, mitomycin C, cisplatin, carboplatin, oxaliplatin and docetaxel.

In embodiments, response to therapy is measured using the methods provided herein (e.g., through molecular characterization of circulating multiple myeloma cells). In embodiments, response to therapy is measured by a reduction in M protein levels in serum and/or urine and the reduction in size or disappearance of plasmacytomas. The international uniform response criteria for MM have expanded upon the European Group for Blood and Marrow Transplantation criteria to provide a more comprehensive evaluation system (Durie B. G. et al., Leukemia, 20:1467-73 (2006)). Importantly, achievement of response has been associated with improved survival in SCT trials with high-dose therapy. Similarly, time to progression (TTP) has been shown to be an important surrogate for improved survival. Despite high response rates to frontline therapy, virtually all patients eventually relapse. Table 2 shows the international uniform response criteria for MM.

TABLE 2
International uniform response criteria for multiple myeloma (MM)

| Response | |
|---|---|
| Subcategory | Response Criteria |
| CR (complete | Negative immunofixation on the serum and urine and disappearance of |
| response) | any soft tissue plasmacytomas and ≤5% plasma cells in bone marrow |
| sCR (stringent | CR as described above, plus: |
| complete response) | normal free light chain (FLC) ratio and absence of clonal cells in bone |
| | marrow by immunohistochemistry or immunofluorescence |
| VGPR (very good | Serum and urine M-protein detectable by immunofluorescence but not |
| partial response) | on electrophoresis or 90% or greater reduction in serum M-protein plus |
| | urine M-protein level <100 mg per 24 hours |
| PR (partial | ≥50% reduction of serum M-protein and reduction in 24-h urinary M- |
| response) | protein by ≥90% or to <200 mg per 24 h |
| | If the serum and urine M-protein are unmeasurable, a ≥50% decrease |
| | in the difference between involved and uninvolved FLC levels is |
| | required in place of the M-protein criteria |
| | If serum and urine M-protein are unmeasurable, and serum free light |
| | assay is also unmeasurable, ≥50% reduction in plasma cells is required |
| | in place of M-protein, provided baseline bone marrow plasma cell |
| | percentage was ≥30% |
| | In addition to the above listed criteria, if present at baseline, a ≥50% |
| | reduction in the size of soft tissue plasmacytomas is also required. |
| SD (stable disease) | Not meeting criteria for CR, VGPR, PR or progressive disease |

In embodiments, the subject has been diagnosed with cancer or is at risk of developing a multiple myeloma.

For therapeutic use, administration of an agent can begin at the detection or surgical removal of tumors. This can be followed by boosting doses until at least symptoms are substantially abated and for a period thereafter.

The pharmaceutical compositions for therapeutic treatment are intended for parenteral, topical, nasal, oral or local administration. Preferably, the pharmaceutical compositions are administered parenterally, e.g., intravenously, subcutaneously, intradermally, or intramuscularly. The disclosure provides compositions for parenteral administration which comprise a solution of a suitable agent dissolved or suspended in an acceptable carrier, preferably an aqueous carrier. A variety of aqueous carriers may be used, e.g., water, buffered water, saline, glycine, hyaluronic acid, and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents, and the like, for example, sodium acetate, sodium lactate, sodium chloride, potassium chloride, calcium chloride, sorbitan monolaurate, triethanolamine oleate, etc.

In an embodiment, the cancer therapeutic is an immunotherapeutic (e.g., an antibody). The cancer therapeutic can be a chimeric antigen receptor (CAR) T cell. The immunotherapeutic may be a cytokine therapeutic (such as an interferon or an interleukin), a dendritic cell therapeutic or an antibody therapeutic, such as a monoclonal antibody. In an embodiment, the immunotherapeutic is a neoantigen (see, e.g., U.S. Pat. No. 9,115,402 and US Patent Publication Nos. 20110293637, 20160008447, 20160101170, 20160331822 and 20160339090).

In some embodiments, the disease is MM or NDMM, and may be treated with any treatment known in the art as effective in treating NDMM or MM. In some embodiments, such effective treatment for MM or NDMM is a three-drug regimen, or a four-drug regimen. In some embodiments, the treatment effective for treating MM or NDMM includes an immunomodulatory agent (IMiD), a proteasome inhibitor (PI), and a corticosteroid. Exemplary immunomodulatory agents include: Empliciti™ [elotuzumab], Thalomid® [thalidomide], Pomalyst™ [pomalidomide], or Revlimid® [lenalidomide]. Exemplary proteasome inhibitors include: Velcade® [bortezomib], Ninlaro™ [ixazomib] or Kyprolis™ [carfilzomib]. Exemplary corticosteroids include dexamethasone or prednisone.

In some embodiments, the three-drug regimen is lenalidomide, bortezomib, and dexamethasone, carfilzomib, lenalidomide, and dexamethasone (KRd), or carfilzomib, cyclophosphamide, and dexamethasone. In some embodiments, the treatment effective for treating MM or NDMM includes a CD38 monoclonal antibody. Exemplary CD38 monoclonal antibodies include Darzalex™ [daratumumab] or Sarclisa™ [isatuximab]. In some embodiments, the four-drug regimen includes bortezomib, melphalan, and prednisone or bortezomib, thalidomide, and dexamethasone. In some embodiments, the treatment effective for treating MM or NDMM includes daratumumab.

In some embodiments, the disease is MGUS and/or high-risk MGUS, and may be treated with any treatment known in the art as effective in treating MGUS and/or high-risk MGUS. In some embodiments, the treatment effective for treating MGUS and/or high-risk MGUS is daratumumab.

In some embodiments, the disease is SMM and/or high-risk SMM, and may be treated with any treatment known in the art as effective in treating SMM and/or high-risk SMM. In some embodiments, the treatment effective in treating SMM and/or high-risk SMM is lenalidomide and dexamethasone. In some embodiments, the treatment effective in treating SMM and/or high-risk SMM includes any treatment disclosed herein as effective in treating MM or NDMM. In some embodiments, the treatment effective in treating SMM and/or high-risk SMM is daratumumab and/or isatuximab.

### Hardware and Software

The present disclosure also relates to a computer system involved in carrying out the methods of the disclosure relating to both computations and detection of marker binding to a capture molecule. The methods described herein, analyses can be performed on general-purpose or specially-programmed hardware or software. One can then record the results (e.g., characterization of MGUS, SMM, or MM in a biological sample, or determination of risk of disease progression) on tangible medium, for example, in computer-readable format such as a memory drive or disk or simply printed on paper, displayed on a monitor (e.g., a computer screen, a smart device, a tablet, a television screen, or the like), or displayed on any other visible medium. The results also could be reported on a computer screen.

In aspects, the analysis is performed by an algorithm. The analysis of capture molecule binding will generate results that are subject to data processing. Data processing can be performed by the algorithm. One of ordinary skill can readily select and use the appropriate software and/or hardware to analyze a sequence.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

Data can be analyzed with the use of a programmable digital computer. The computer program analyzes the sequence data to indicate alterations (e.g., alterations in expression for any marker or combinations of markers disclosed herein) observed in the data. In aspects, software used to analyze the data can include code that applies an algorithm to the analysis of the results. The software also can also use input data (e.g., sequence) to characterize a biological sample.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation (e.g., sequencing results) can be displayed on a monitor, display, or other visualizable medium (e.g., a printout). In some embodiments, data or the results of a calculation may be presented in an auditory form.

### Kits

The disclosure also provides kits for use in embodiments of the methods provided herein. Kits of the instant disclosure may include one or more containers comprising one or more capture agents and/or articles of manufacture suitable for use in detecting markers in a subject and/or for treatment of a multiple myeloma (MM), such as newly diagnosed multiple myeloma (NDMM), or a multiple myeloma precursor disease, such as monoclonal gammopathy of undetermined significance (MGUS) or smoldering multiple myeloma (SMM). In some embodiments, the kits further include instructions for use in accordance with the methods of this disclosure. In some embodiments, these instructions comprise a description of use of the one or more capture agents and/or article of manufacture for use in detecting markers disclosed herein and/or use of an agent for treatment of a multiple myeloma (MM), such as newly diagnosed multiple myeloma (NDMM), or a multiple myeloma precursor disease, such as monoclonal gammopathy of undetermined significance (MGUS) or smoldering multiple myeloma (SMM).

Instructions supplied in the kits of the instant disclosure can be written instructions on a label or package insert (e.g., a paper sheet included in the kit), or machine-readable instructions (e.g., instructions carried on a magnetic or optical storage disk). Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. For example, the kit comprises a container and a label or package insert(s) on or associated with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

Described below are examples of ways in which techniques described herein may be implemented. It should be appreciated that these examples are merely illustrative, that embodiments are not limited to operating in accordance with the specific examples shown in the figures and discussed below, and that other embodiments are possible.

### EXAMPLES

### Example 1: High-Throughput Plasma Proteomics to Define the Precursor Multiple Myeloma Proteome and Identify Candidate High-Risk Disease Biomarkers of Progression

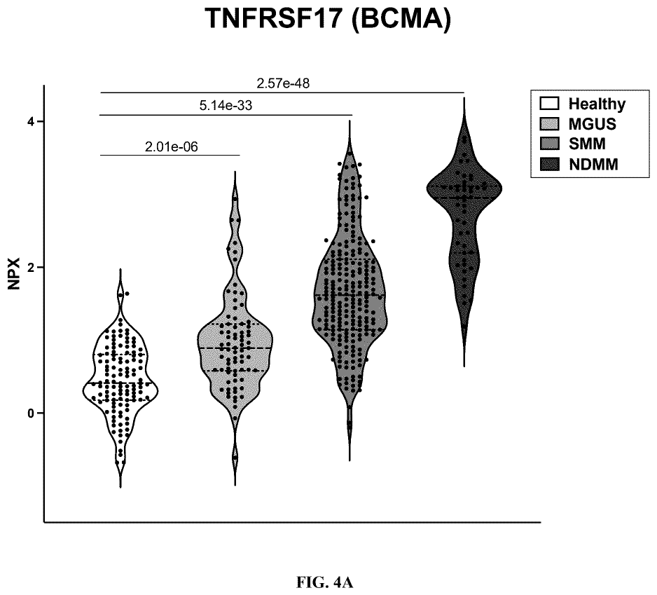

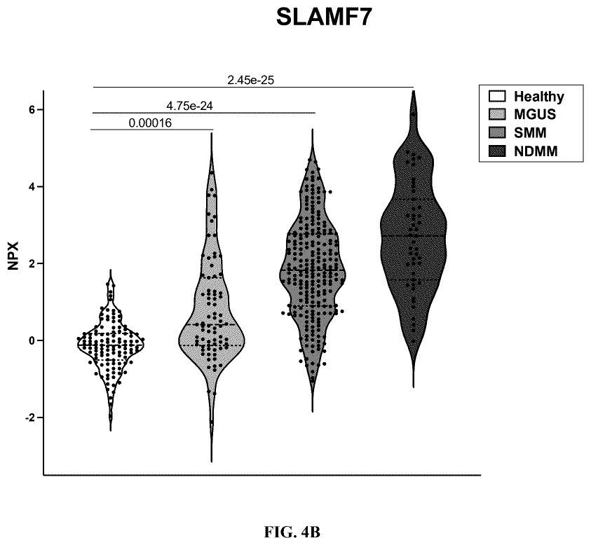

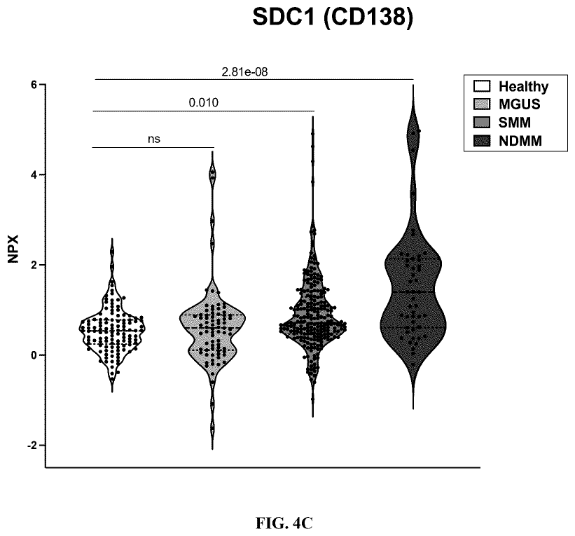

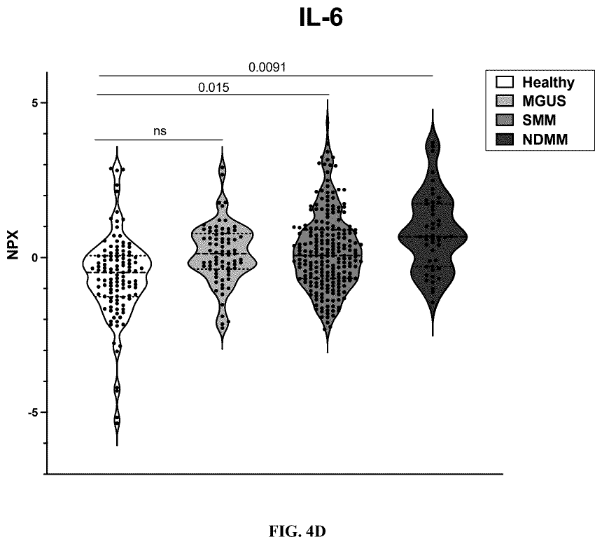

High-throughput plasma proteomic profiling for approximately 3000 proteins was carried out simultaneously using the Olink® Explore 3072 library and Proximity Extension Assay (PEA) technology (FIGS. 4A-4D). Targeted proteins were recognized by multiplexed, matched pairs of antibodies labelled with unique DNA oligonucleotides that upon binding came into proximity, hybridized, and were extended to generate a unique sequence for protein identification with NextGen DNA sequencing.

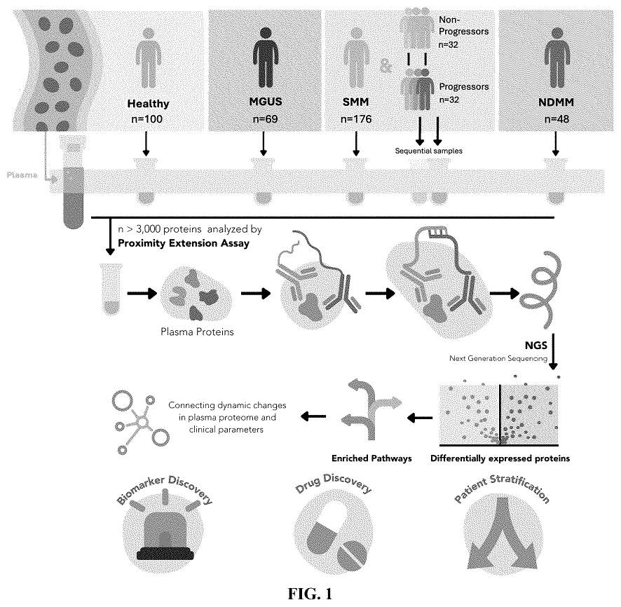

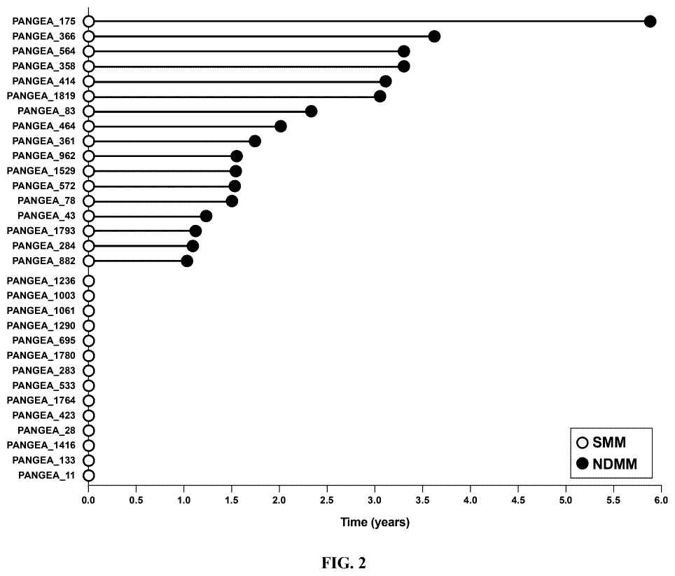

A total of 465 peripheral blood (PB) plasma samples from 393 individuals, including monoclonal gammopathy of undetermined significance (MGUS) (n=69), smoldering multiple myeloma (SMM) (n=176), multiple myeloma (MM) (n=48), and healthy controls (n=100) were profiled (FIG. 1). Sequential samples from patients with progressive disease (n=27) and patients with stable disease with matched clinical follow-up time were also profiled (FIG. 2). Precursor defined samples from progressors ranged 1.03-5.88 yrs prior to diagnosis and were untreated in the precursor setting, while MM disease samples were also collected prior to any active disease therapy. Patients had a median clinical follow-up time of 7.05 years. T-tests, ANOVAs, and a linear mixed effect (LME) model were used to identify proteins that change across disease stages, progression status, and time. Results were adjusted for multiple testing using the Benjamini-Hochberg Method.

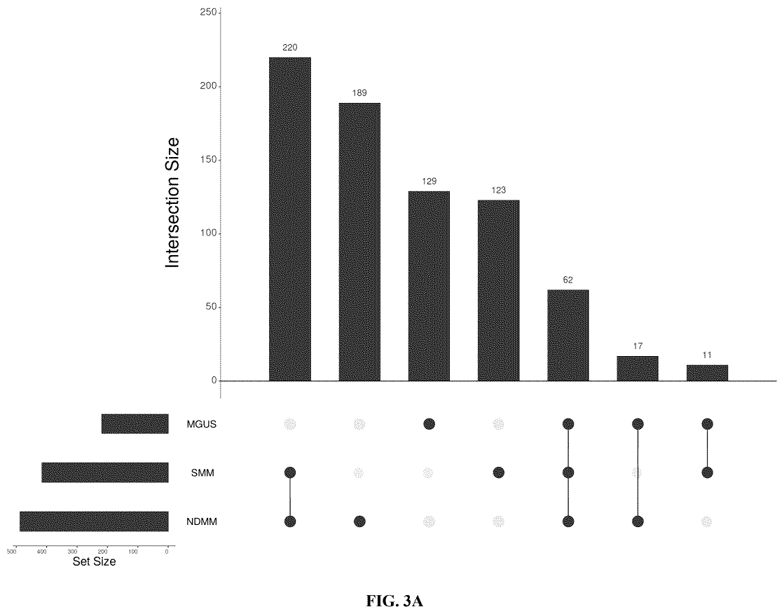

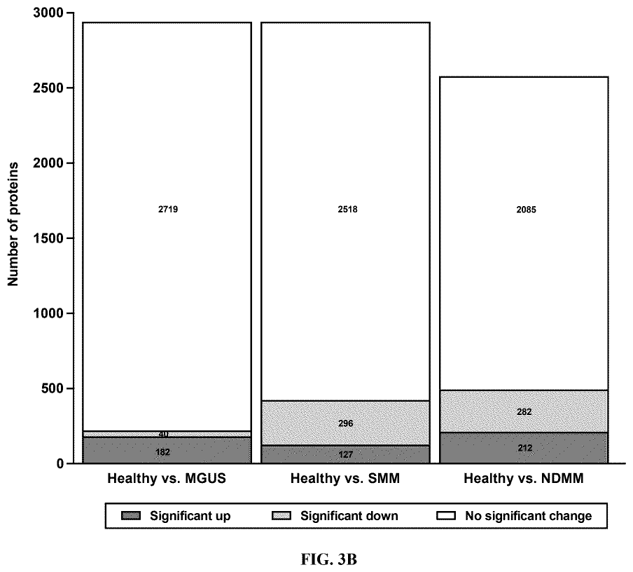

751 significantly dysregulated proteins were identified, with the majority upregulated in progressive disease (FIGS. 3A-3B). Proteins that significantly distinguished MGUS, SMM, and NDMM from healthy donors (n=222, 423 and 494) were identified, where increasing B-cell maturation antigen (TNFRSF17/BCMA) was a significant classifier and positive control. Tables 1-4 below, list proteins that significantly distinguished MGUS, SMM, and NDMM from healthy donors (Tables 1-3), as well as proteins that distinguished low risk SMM from high-risk SMM (Table 4).

TABLE 1
Genes With Significant Alteration in Expression
in MGUS Compared to Healthy Subjects

| Gene | MGUS.vs.Normal_logFC | P value |
|---|---|---|
| CMIP | 1.970570739 | 0.046107533 |
| FKBP1B | 1.917628433 | 0.045201688 |
| SERPINH1 | 1.497100973 | 0.036471375 |
| ALDH5A1 | 1.492837576 | 0.000727763 |
| VASH1 | 1.417484051 | 0.033915559 |
| RBPMS2 | 1.404004389 | 0.005231664 |
| FCRLB | 1.395277448 | 0.001350864 |
| PRKG1 | 1.388320702 | 0.018595685 |
| MESD | 1.3864822 | 0.018595685 |
| GRAP2 | 1.366101601 | 0.04106391 |
| SRC | 1.340033212 | 0.025562604 |
| DNAJB6 | 1.33139569 | 0.033648134 |
| RHOC | 1.285364292 | 0.021717822 |
| MYDGF | 1.25778173 | 0.006545361 |
| VAV3 | 1.256586774 | 0.036180689 |
| APPL2 | 1.254671122 | 0.014799685 |
| MANF | 1.229772776 | 0.037487722 |
| PHACTR2 | 1.216559526 | 0.034169987 |
| PDE5A | 1.215652613 | 0.011410218 |
| DNAJA2 | 1.191735853 | 0.048143389 |
| DTD1 | 1.183352746 | 0.028542645 |
| AIFM1 | 1.18285749 | 0.015862379 |
| BCL2L1 | 1.178078561 | 0.017221221 |
| SPRY2 | 1.174896592 | 0.022891238 |
| LDLRAP1 | 1.171293097 | 0.028542645 |
| CDC42BPB | 1.171078291 | 0.017221221 |
| YES1 | 1.167405942 | 0.026150931 |
| PDLIM7 | 1.16675566 | 0.04127052 |
| VTI1A | 1.161930557 | 0.017221221 |
| SLA2 | 1.158874224 | 0.014583526 |
| CLIP2 | 1.151204126 | 0.021342627 |
| EHD3 | 1.147065533 | 0.027719399 |
| ERP29 | 1.13869844 | 0.031219598 |
| YARS1 | 1.130095686 | 0.049434697 |
| GIPC3 | 1.127730124 | 0.026150931 |
| TXNDC5 | 1.124471518 | 0.000184865 |
| STXBP1 | 1.123627985 | 0.045201688 |
| SNCA | 1.11733772 | 0.046313252 |
| CA13 | 1.115917043 | 0.033888339 |
| SARG | 1.114957078 | 0.03141683 |
| DAB2 | 1.112004362 | 0.027898797 |
| ITGB1BP2 | 1.107191692 | 0.049816775 |
| NDUFB7 | 1.093320671 | 0.005272823 |
| CETN3 | 1.091418367 | 0.048143389 |
| SH2B3 | 1.089165926 | 0.020273315 |
| FKBP14 | 1.079510227 | 0.024007 |
| DECR1 | 1.067166024 | 0.020500354 |
| ARHGEF12 | 1.06698 | 0.021287244 |
| CDKN1A | 1.063479854 | 0.035618036 |
| TRIM25 | 1.06067457 | 0.02563193 |
| LONP1 | 1.056048401 | 0.018711057 |
| DOK2 | 1.054817621 | 0.046313252 |
| KHK | 1.047246727 | 0.000157857 |
| SLAMF7 | 1.04464924 | 0.000157857 |
| SMTN | 1.028286708 | 0.015862379 |
| ACADSB | 1.023922134 | 0.014799685 |
| CMC1 | 1.019900711 | 0.036481142 |
| PKN3 | 1.014572942 | 0.032856213 |
| BANK1 | 1.014444917 | 0.033648134 |
| ECHS1 | 1.014112899 | 0.018711057 |
| NCK2 | 1.012417716 | 0.042420248 |
| NRGN | 1.003045234 | 0.022891238 |
| SLAMF1 | 0.998835274 | 0.014799685 |
| DNM1 | 0.997409518 | 0.04127052 |
| EIF2AK3 | 0.994139931 | 0.011263448 |
| DNAJC6 | 0.991753053 | 0.04127052 |
| CLPP | 0.982434495 | 0.025500914 |
| TBC1D23 | 0.981423313 | 0.027898797 |
| SLMAP | 0.971822065 | 0.021610938 |
| OPHN1 | 0.971622154 | 0.042369421 |
| PDIA4 | 0.968430353 | 0.005272823 |
| PRKAR1A | 0.965293227 | 0.032856213 |
| CCL13 | 0.963573858 | 0.017221221 |
| AKR7L | 0.962535022 | 0.000298622 |
| TBC1D5 | 0.960797611 | 0.04228248 |
| PPIB | 0.955404919 | 0.027719399 |
| PDLIM5 | 0.950824729 | 0.035618036 |
| CRACR2A | 0.942142186 | 0.047674096 |
| SDCCAG8 | 0.937862811 | 0.045201688 |
| EIF4G3 | 0.937765356 | 0.049069599 |
| WASF1 | 0.936823486 | 0.045201688 |
| PLA2G4A | 0.93458895 | 0.04228248 |
| EEF1D | 0.934473715 | 0.034289973 |
| NT5C | 0.934160083 | 0.033648134 |
| PNMA1 | 0.932886628 | 0.014799685 |
| CRYZL1 | 0.92058477 | 0.0400038 |
| KAZN | 0.912348715 | 0.046313252 |
| CACNB3 | 0.910628526 | 0.045116375 |
| PMM2 | 0.900231269 | 0.033648134 |
| KIF22 | 0.893440811 | 0.036481142 |
| METAP1D | 0.890991648 | 0.038442962 |
| MTSS2 | 0.885555761 | 0.045825232 |
| ZNRD2 | 0.88232146 | 0.011410218 |
| MMUT | 0.880284756 | 0.037073367 |
| DUT | 0.869165021 | 0.045201688 |
| MTSS1 | 0.862901545 | 0.047632724 |
| MAP2 | 0.857813876 | 0.01599954 |
| TNFRSF13B | 0.855898586 | 0.000473081 |
| TXNDC9 | 0.848295965 | 0.045201688 |
| VAMP8 | 0.844324845 | 0.045201688 |
| SERPINE1 | 0.843205024 | 0.045201688 |
| MYO6 | 0.83148853 | 0.000473081 |
| TJAP1 | 0.82517115 | 0.04106391 |
| FUOM | 0.822669229 | 0.011410218 |
| NOS2 | 0.822432223 | 0.000157857 |
| NFX1 | 0.81809968 | 0.027898797 |
| DGKZ | 0.816683844 | 0.021717822 |
| TPPP2 | 0.81657181 | 0.034289973 |
| PACS2 | 0.813986703 | 0.045201688 |
| L3HYPDH | 0.812319418 | 0.046313252 |
| TOMM20 | 0.811932324 | 0.010767686 |
| HSPB1 | 0.80510416 | 0.017221221 |
| ATG16L1 | 0.796562615 | 0.028542645 |
| ESYT2 | 0.795342089 | 0.048466401 |
| TRAF2 | 0.788155329 | 0.033648134 |
| MARS1 | 0.783811985 | 0.048632173 |
| HEXIM1 | 0.780989932 | 0.045201688 |
| RGS10 | 0.768674458 | 0.048999373 |
| TDO2 | 0.761661055 | 0.017221221 |
| CRYM | 0.758442728 | 0.027898797 |
| ENO2 | 0.757037021 | 0.031121863 |
| ENO3 | 0.722283121 | 0.021287244 |
| CNPY4 | 0.70817259 | 0.045116375 |
| LETM1 | 0.701450198 | 0.033648134 |
| TNFRSF17 | 0.698651177 | 2.01E−06 |
| IQGAP2 | 0.694616523 | 0.04127052 |
| OTUD7B | 0.688241024 | 0.031075252 |
| UROD | 0.6873173 | 0.049769726 |
| TANK | 0.686844388 | 0.046313252 |
| IMMT | 0.685597746 | 0.001908809 |
| CRELD2 | 0.67806996 | 0.015923815 |
| PBLD | 0.677138551 | 0.002941279 |
| FCRL5 | 0.675442053 | 0.021342627 |
| ECHDC3 | 0.674563844 | 0.023202981 |
| DCTN1 | 0.656131612 | 0.046313252 |
| LGALS8 | 0.654029431 | 0.045201688 |
| HTRA2 | 0.652436102 | 0.037503597 |
| MMP7 | 0.638964917 | 0.030663517 |
| ITM2A | 0.638522496 | 0.003406342 |
| EHBP1 | 0.632229863 | 0.047072985 |
| MRPL58 | 0.628581392 | 0.031121863 |
| DNM3 | 0.618348438 | 0.027898797 |
| RAB3GAP1 | 0.616127339 | 0.017221221 |
| CA5A | 0.615719502 | 0.045201688 |
| CD79B | 0.604455653 | 0.027018047 |
| SCPEP1 | 0.598124472 | 0.031958907 |
| CSF3 | 0.565126292 | 0.018740872 |
| FDX2 | 0.562673731 | 0.046764955 |
| MOCS2 | 0.547678632 | 0.034289973 |
| CD40 | 0.546598467 | 0.027719399 |
| BSND | 0.54025465 | 0.00391828 |
| GTF2IRD1 | 0.53791142 | 0.048369294 |
| GRPEL1 | 0.536240282 | 0.037073367 |
| PDIA3 | 0.533842287 | 0.000625337 |
| CD274 | 0.533074511 | 0.037464591 |
| CDNF | 0.531804881 | 0.002697134 |
| GSAP | 0.531648254 | 0.033406583 |
| THBS2 | 0.529265417 | 0.011897727 |
| VSIG2 | 0.528998667 | 0.033313409 |
| MRI1 | 0.527633061 | 0.028542645 |
| JAM3 | 0.518301879 | 0.034289973 |
| PLXNB3 | 0.488862837 | 0.046313252 |
| BTN3A2 | 0.481253316 | 0.004386027 |
| ART5 | 0.47422822 | 0.032856213 |
| SUMF2 | 0.443290541 | 0.033648134 |
| SOD2 | 0.427646673 | 0.018711057 |
| DCXR | 0.408897924 | 0.045825232 |
| TIMM10 | 0.39808371 | 0.045201688 |
| LPL | 0.37024137 | 0.045116375 |
| P4HB | 0.368032675 | 0.035912982 |
| SCGB1A1 | 0.363834428 | 0.037274584 |
| FGA | 0.362258738 | 0.048466401 |
| OCLN | 0.361686664 | 0.048143389 |
| ERN1 | 0.344539779 | 0.046313252 |
| ADA2 | 0.336542473 | 0.033313409 |
| SEL1L | 0.324211623 | 0.04106391 |
| B2M | 0.290728942 | 0.027719399 |
| LRIG3 | 0.274142462 | 0.034018575 |
| LGMN | 0.251432615 | 0.049141092 |
| JAM2 | 0.235978132 | 0.048999373 |
| CD46 | 0.230437455 | 0.033648134 |
| PSAP | 0.22282369 | 0.045201688 |
| TNFSF13B | −0.297873187 | 0.025500914 |
| WFDC1 | −0.32744693 | 0.018711057 |
| CD22 | −0.424504395 | 0.04996662 |
| CD6 | −0.440673883 | 0.045201688 |
| RNF43 | −0.461144125 | 0.031958907 |
| DEFB116 | −0.473398389 | 0.028270879 |
| FCN1 | −0.478291177 | 0.004386027 |
| PDCL2 | −0.48859654 | 0.043213472 |
| MORF4L2 | −0.502486251 | 0.041281435 |
| TGFA | −0.506874674 | 0.018711057 |
| GPD1 | −0.516866215 | 0.008344816 |
| CLEC2L | −0.558113691 | 0.027719399 |
| NAPRT | −0.581621207 | 0.040785024 |
| MMP9 | −0.611293909 | 0.018711057 |
| IL1RN | −0.627263684 | 0.048143389 |
| NECTIN1 | −0.627772224 | 0.017221221 |
| TNFSF14 | −0.68263621 | 0.036481142 |
| HCLS1 | −0.711105897 | 0.043213472 |
| OLR1 | −0.734806469 | 0.019427719 |
| CNTNAP4 | −0.750244118 | 0.033648134 |
| PDE4D | −0.795561246 | 0.00391828 |
| IFNL1 | −0.803578549 | 0.002941279 |
| IL18RAP | −0.854771131 | 0.041724832 |
| NPHS2 | −0.870378214 | 0.043402346 |
| DCUN1D1 | −0.959290713 | 0.025500914 |
| S100A11 | −0.965867308 | 0.002941279 |
| COL9A2 | −0.99508001 | 0.021611591 |
| S100A12 | −1.01909142 | 0.021342627 |
| RBP7 | −1.089505226 | 0.002169998 |
| DAPK2 | −1.099721798 | 0.045201688 |
| BCL2L15 | −1.122816911 | 0.010006354 |
| RAB44 | −1.141532049 | 0.021342627 |
| IDO1 | −1.159903648 | 0.003150067 |
| IDO1 | −1.22512477 | 0.002697134 |
| NCF2 | −1.250122962 | 0.021717822 |
| CLC | −1.313677692 | 0.002169998 |
| IDO1 | −1.329346643 | 0.002511749 |
| IDO1 | −1.358145324 | 0.002169998 |
| RASGRF1 | −1.762589843 | 0.003746024 |
| CASP1 | −1.92131288 | 0.008433347 |

TABLE 2
Genes With Significant Alteration in Expression
in SMM Compared to Healthy Subjects

| Gene | SMM.vs.Normal_logFC | P value |
|---|---|---|
| FCRLB | 2.299139241 | 3.17E−15 |
| SLAMF7 | 1.92722551 | 4.75E−24 |
| TNFRSF13B | 1.511379969 | 1.43E−19 |
| SLAMF1 | 1.484272598 | 1.36E−08 |
| FCRL5 | 1.414622456 | 3.85E−14 |
| IL5RA | 1.392244591 | 3.57E−09 |
| IL5 | 1.302310262 | 0.039446494 |
| TNFRSF17 | 1.287782833 | 5.14E−33 |
| CD79B | 1.114576325 | 1.93E−10 |
| SPINT3 | 1.081222949 | 0.045323298 |
| CNTN5 | 1.001918816 | 9.14E−10 |
| AKR7L | 0.934101434 | 2.81E−06 |
| CSF3 | 0.930432636 | 2.11E−09 |
| FUOM | 0.896887337 | 0.000125506 |
| TXNDC5 | 0.856631627 | 0.000110164 |
| MZB1 | 0.832280379 | 1.31E−07 |
| PRDX1 | 0.796286552 | 0.009537413 |
| NOS2 | 0.777704747 | 1.87E−06 |
| ZNRD2 | 0.77717168 | 0.001453201 |
| ITM2A | 0.760519797 | 2.60E−07 |
| EPO | 0.740819039 | 0.002961018 |
| TDO2 | 0.739178559 | 0.001735919 |
| KHK | 0.727223325 | 0.000335658 |
| HAO1 | 0.70792746 | 0.019680038 |
| PDZK1 | 0.707868414 | 0.000944978 |
| KIF22 | 0.693700515 | 0.033166352 |
| APLP1 | 0.68104098 | 1.18E−06 |
| IL6 | 0.671684634 | 0.006703542 |
| THBS2 | 0.664200271 | 6.69E−06 |
| IL6 | 0.651545095 | 0.002913206 |
| FCRL2 | 0.647449757 | 0.002360004 |
| ICAM3 | 0.645748661 | 1.92E−10 |
| NDUFB7 | 0.63723555 | 0.0385118 |
| CHGA | 0.634549942 | 0.009226093 |
| QPCT | 0.618657182 | 1.47E−08 |
| ADGRG1 | 0.608230176 | 0.00657671 |
| MMP7 | 0.588771433 | 0.004943673 |
| ENO3 | 0.572905813 | 0.017699517 |
| RALB | 0.567719554 | 0.008003467 |
| KLK13 | 0.560396174 | 0.000572117 |
| TEX101 | 0.551510399 | 0.033624859 |
| ADA2 | 0.548200318 | 8.45E−08 |
| IL6 | 0.546618939 | 0.014718206 |
| CEACAM16 | 0.538338305 | 0.022558808 |
| IL6 | 0.536618016 | 0.02433165 |
| MYO6 | 0.532905046 | 0.005385673 |
| CRELD2 | 0.527214334 | 0.008731558 |
| ADH1B | 0.525969923 | 0.002889036 |
| FTCD | 0.517261929 | 0.005638261 |
| RAB3GAP1 | 0.512064905 | 0.00474772 |
| LY9 | 0.504186074 | 0.000155044 |
| KRT18 | 0.500675424 | 0.031597246 |
| CD48 | 0.496841999 | 1.15E−07 |
| IMMT | 0.491784676 | 0.002360004 |
| LAG3 | 0.477900838 | 0.000511709 |
| ART5 | 0.47771159 | 0.002223393 |
| EFEMP1 | 0.469028912 | 0.000894691 |
| MILR1 | 0.467353814 | 0.004943673 |
| CD274 | 0.465409398 | 0.013557927 |
| UPB1 | 0.462648805 | 0.034302311 |
| IGFBP2 | 0.453224087 | 0.011562636 |
| PROK1 | 0.442069095 | 0.015069934 |
| FGA | 0.435003319 | 0.001670086 |
| LILRB4 | 0.424500932 | 0.002889036 |
| RARRES1 | 0.421503253 | 0.045567438 |
| CDCP1 | 0.42017103 | 0.014151138 |
| BSND | 0.418646196 | 0.002166365 |
| AGXT | 0.414248614 | 0.032190831 |
| LAMP3 | 0.411218981 | 0.017224583 |
| ANXA10 | 0.409684214 | 0.028603965 |
| CKB | 0.408920606 | 0.019251953 |
| CEACAM19 | 0.404722887 | 0.007304806 |
| SDC1 | 0.401639022 | 0.010318188 |
| GDF15 | 0.400723176 | 0.000466978 |
| IGLC2 | 0.398098365 | 0.026103239 |
| VSIG10L | 0.380916037 | 0.033696048 |
| SORBS1 | 0.379771301 | 0.014859173 |
| ATP5F1D | 0.378609295 | 0.045597025 |
| GALNT3 | 0.375278043 | 0.007255156 |
| B3GAT3 | 0.372435798 | 0.033624859 |
| FGL1 | 0.352596451 | 0.01377036 |
| REG3A | 0.349465553 | 0.041224471 |
| NCR3LG1 | 0.345270935 | 0.002502687 |
| LPL | 0.344949849 | 0.010144064 |
| CAPS | 0.344064976 | 0.030631379 |
| RBFOX3 | 0.339454286 | 0.010856019 |
| GIPC2 | 0.331518977 | 0.003189519 |
| SRPX | 0.330604018 | 0.019182576 |
| SCGB1A1 | 0.324819625 | 0.010899334 |
| YAP1 | 0.324772128 | 0.003579252 |
| DCXR | 0.315812856 | 0.045597025 |
| B2M | 0.314456708 | 0.000507326 |
| OCLN | 0.309133802 | 0.026103239 |
| WFDC2 | 0.302554507 | 0.022157346 |
| NPTX2 | 0.299598937 | 0.000629825 |
| IL15 | 0.29683713 | 0.045323298 |
| ACP5 | 0.28826737 | 0.000589107 |
| SHBG | 0.286838316 | 0.016286529 |
| NTF4 | 0.284936859 | 0.034972897 |
| BTN3A2 | 0.282549062 | 0.033624859 |
| SEL1L | 0.281078657 | 0.016179984 |
| PCDH17 | 0.275850324 | 0.01195849 |
| ST6GAL1 | 0.272413146 | 0.022193762 |
| TINAGL1 | 0.271008869 | 1.86E−05 |
| PDIA3 | 0.268955141 | 0.033624859 |
| ADGRE2 | 0.265288441 | 0.000724614 |
| ADGRE5 | 0.264717581 | 0.001670086 |
| PLA2G15 | 0.257244805 | 0.002879069 |
| C1QTNF5 | 0.255839347 | 0.002223393 |
| C7 | 0.254800213 | 0.000166522 |
| RNASET2 | 0.253787837 | 0.009935213 |
| LTBP2 | 0.244146988 | 0.03703706 |
| ICAM2 | 0.234402101 | 0.003235519 |
| ITGBL1 | 0.219071256 | 0.039021131 |
| ADAMTSL4 | 0.21770675 | 0.013783311 |
| AMY1A_AMY1B_AMY1C | 0.215120967 | 0.017289407 |
| IL18BP | 0.213972778 | 0.019585701 |
| LGMN | 0.21176344 | 0.03079944 |
| RELT | 0.198591568 | 0.045597025 |
| NRCAM | 0.195560822 | 0.035356078 |
| SEMA3F | 0.187571357 | 0.018631803 |
| IGFBP7 | 0.185962042 | 0.016992959 |
| NPC2 | 0.185349314 | 0.045597025 |
| MRC1 | 0.174880364 | 0.022007417 |
| CRIM1 | 0.167204114 | 0.006154538 |
| ANGPTL1 | 0.161995447 | 0.047512304 |
| BMPER | 0.143226541 | 0.016647533 |
| A1BG | −0.114013983 | 0.014595011 |
| SERPING1 | −0.117378438 | 0.004773197 |
| TF | −0.118691514 | 0.034240917 |
| SERPINF2 | −0.121812232 | 0.006835614 |
| AFM | −0.130768188 | 0.019273136 |
| HEPH | −0.131179278 | 0.039160407 |
| LAMP2 | −0.133434564 | 0.02726174 |
| ROBO2 | −0.134517918 | 0.035084751 |
| APOA1 | −0.136828295 | 0.012958391 |
| SERPINA6 | −0.137209408 | 0.016389278 |
| RECK | −0.137923911 | 0.016580582 |
| CA4 | −0.139077144 | 0.039021131 |
| FAP | −0.139372372 | 0.035739304 |
| ATRN | −0.141306626 | 0.001040647 |
| EGFR | −0.145998253 | 0.001393261 |
| CPB2 | −0.148268689 | 0.006553767 |
| ERBB3 | −0.151108715 | 0.002961018 |
| TFPI | −0.153589034 | 0.03931767 |
| MEGF9 | −0.154726773 | 0.004847066 |
| CRLF1 | −0.158131017 | 0.023024536 |
| CLU | −0.158713444 | 0.0019669 |
| ERBB2 | −0.166217274 | 0.001917766 |
| IGDCC4 | −0.16714066 | 0.008704926 |
| DPP4 | −0.172513412 | 0.011290843 |
| PI16 | −0.173242032 | 0.039160407 |
| ORM1 | −0.177354608 | 0.011642841 |
| CD1C | −0.18214094 | 0.017139737 |
| CD34 | −0.186107803 | 0.004232564 |
| BST1 | −0.192963277 | 0.028388481 |
| VAT1 | −0.19473843 | 0.040407836 |
| TCN1 | −0.196041082 | 0.0339295 |
| KLKB1 | −0.199991926 | 0.000642031 |
| PLAUR | −0.20023939 | 0.036881094 |
| LUZP2 | −0.204228178 | 0.043032821 |
| CDON | −0.206651189 | 0.002696648 |
| CRISP3 | −0.206802253 | 0.000487843 |
| ADGRG2 | −0.211706391 | 0.009173877 |
| PTPRC | −0.212260526 | 0.003483889 |
| NPR1 | −0.214803538 | 0.017433967 |
| AKAP12 | −0.217601993 | 0.002889036 |
| IGFBP3 | −0.223777623 | 0.004692136 |
| PLXDC2 | −0.228977208 | 0.009173877 |
| GHR | −0.229750423 | 0.005881827 |
| CR1 | −0.23024967 | 0.005848432 |
| ICAM5 | −0.245374593 | 0.04179858 |
| TRAF3IP2 | −0.253010795 | 0.035189417 |
| HJV | −0.258469179 | 0.039021131 |
| CR2 | −0.262346696 | 0.031310395 |
| PDGFC | −0.262979486 | 0.002696648 |
| WFIKKN1 | −0.265435018 | 0.033624859 |
| GP5 | −0.265848801 | 0.011562636 |
| SEMA4D | −0.268675329 | 0.014859173 |
| CBLN4 | −0.271236004 | 0.022007417 |
| MED21 | −0.27667515 | 0.007200299 |
| BAG3 | −0.277931145 | 0.014240034 |
| HMOX1 | −0.278720382 | 0.028603965 |
| PRG3 | −0.280679778 | 0.047184112 |
| C1S | −0.282901748 | 1.36E−05 |
| SELL | −0.283381355 | 6.21E−06 |
| GRIK2 | −0.285003046 | 0.00474772 |
| FCRL1 | −0.285520912 | 0.008877626 |
| FGFBP2 | −0.293456886 | 0.005724594 |
| CPOX | −0.299052372 | 0.000572117 |
| CD101 | −0.304364776 | 0.015221768 |
| LTB | −0.309680721 | 0.001587193 |
| ITGB2 | −0.311825261 | 0.000237603 |
| ITGA11 | −0.318250166 | 0.004847066 |
| STX7 | −0.318835405 | 0.045597025 |
| LCP1 | −0.322470579 | 0.010094286 |
| NEDD4L | −0.324302287 | 0.03839291 |
| GIMAP7 | −0.335986937 | 0.02739452 |
| ADAM8 | −0.339269934 | 0.002769978 |
| FGFR4 | −0.340085217 | 0.010403107 |
| ITGAM | −0.340329905 | 0.001309269 |
| EVPL | −0.341199227 | 0.046045509 |
| GPC5 | −0.342519166 | 0.03927497 |
| SNED1 | −0.343070717 | 0.002696648 |
| BMP4 | −0.343105306 | 0.019258757 |
| CLINT1 | −0.34446586 | 0.032842914 |
| VNN2 | −0.346112849 | 0.026103239 |
| NMRK2 | −0.349334122 | 0.017511431 |
| KEL | −0.351175816 | 0.002889036 |
| DPEP2 | −0.364370419 | 0.005385673 |
| GIPR | −0.364421392 | 0.045323298 |
| GLI2 | −0.366266199 | 0.046600437 |
| CSTB | −0.369099541 | 0.045597025 |
| CLEC4A | −0.370790516 | 0.003012729 |
| LGALS3 | −0.370821986 | 0.001579465 |
| EZR | −0.371970744 | 0.003012729 |
| CD5L | −0.375327939 | 0.039021131 |
| ARHGAP1 | −0.388876189 | 0.028098572 |
| ODAM | −0.38958941 | 0.038370418 |
| COMMD9 | −0.390372627 | 0.004117179 |
| PDIA5 | −0.391597764 | 0.04179858 |
| IGSF9 | −0.395049427 | 0.039021131 |
| KLRC1 | −0.398213025 | 0.032401947 |
| CCDC50 | −0.400277749 | 0.013682227 |
| AHNAK | −0.407854736 | 0.002705945 |
| RAD51 | −0.41082471 | 0.036064717 |
| TPPP3 | −0.411392923 | 0.004140005 |
| RPGR | −0.417532511 | 0.045597025 |
| PRL | −0.423535577 | 0.01377036 |
| IL20 | −0.433255671 | 0.045567438 |
| BCL7B | −0.435182762 | 0.014595011 |
| CLEC12A | −0.438220722 | 0.000341565 |
| WFDC1 | −0.439300336 | 1.36E−06 |
| SAT2 | −0.439683558 | 0.034240917 |
| IFT20 | −0.450674969 | 0.016654511 |
| ICAM4 | −0.451393605 | 0.002425344 |
| CA7 | −0.458406898 | 0.041338024 |
| NEK7 | −0.466113308 | 0.011530721 |
| DDX39A | −0.468521832 | 0.014859173 |
| CPXM1 | −0.468772802 | 0.011335732 |
| CLEC4C | −0.47131929 | 0.000813484 |
| CLEC5A | −0.473856879 | 6.69E−06 |
| FASLG | −0.480084702 | 8.43E−05 |
| ERMAP | −0.482215297 | 0.003453187 |
| PDCL2 | −0.492468781 | 0.004255793 |
| CLEC4D | −0.495807632 | 0.012081635 |
| SPRING1 | −0.505639371 | 0.000367858 |
| NGFR | −0.506571188 | 0.00039211 |
| ITGA6 | −0.507346472 | 0.006197653 |
| CST7 | −0.513136043 | 0.017102568 |
| TNFSF13B | −0.516409278 | 1.64E−09 |
| DGCR6 | −0.517569806 | 0.008704926 |
| CEACAM8 | −0.527466376 | 0.002828446 |
| ITPR1 | −0.528084727 | 0.002902732 |
| APP | −0.528403011 | 0.010252339 |
| NAGK | −0.530447818 | 0.040142932 |
| TPT1 | −0.535574834 | 0.043372325 |
| IFNL1 | −0.538161046 | 0.007231891 |
| IL31 | −0.540120867 | 0.016580582 |
| FCAR | −0.545063876 | 0.00393564 |
| ERVV−1 | −0.547524835 | 0.010159628 |
| VEGFC | −0.548345747 | 8.18E−05 |
| BPIFB2 | −0.550717998 | 0.002961018 |
| GAGE2A | −0.557660642 | 0.009836883 |
| NID2 | −0.566759632 | 0.001757829 |
| ARL2BP | −0.567247289 | 0.031310395 |
| DEFB116 | −0.567857028 | 0.000287093 |
| REPS1 | −0.567912446 | 0.039021131 |
| CDA | −0.568870537 | 0.000336383 |
| GBA | −0.571507214 | 0.041224471 |
| SKAP1 | −0.576279094 | 0.017323065 |
| MORF4L2 | −0.580194804 | 0.00132025 |
| DHPS | −0.580754709 | 9.04E−05 |
| PZP | −0.58645907 | 0.005130504 |
| FMNL1 | −0.588104155 | 0.034283385 |
| HNMT | −0.599443999 | 0.001071718 |
| NFKBIE | −0.607226573 | 0.007667272 |
| CNTNAP4 | −0.607839284 | 0.028267333 |
| CCN2 | −0.609043719 | 1.78E−05 |
| RNF43 | −0.612966957 | 1.71E−05 |
| HDGF | −0.617588792 | 0.032655111 |
| PAGR1 | −0.618776218 | 0.010427953 |
| KLF4 | −0.623973385 | 0.007844081 |
| SERPINE2 | −0.624501504 | 0.009173877 |
| NFYA | −0.627093819 | 0.034376667 |
| CD6 | −0.631391967 | 1.78E−05 |
| ERI1 | −0.635144161 | 0.011132775 |
| LBR | −0.637947688 | 0.048849357 |
| PGD | −0.643199585 | 0.004117179 |
| VSTM1 | −0.64567803 | 5.47E−05 |
| PRKRA | −0.657888841 | 0.007836342 |
| COL24A1 | −0.659222413 | 0.008890158 |
| PCBP2 | −0.659742902 | 0.035356078 |
| NAPRT | −0.664202662 | 0.001393261 |
| MSRA | −0.666151028 | 0.030142956 |
| GPD1 | −0.670366025 | 1.84E−07 |
| GLRX | −0.677070314 | 0.000634487 |
| CEP164 | −0.677584383 | 0.002904721 |
| MAP2K1 | −0.683831251 | 0.043485549 |
| PAG1 | −0.68604303 | 0.010969634 |
| GPI | −0.694096363 | 0.00553214 |
| TBCC | −0.696346372 | 0.037166572 |
| MMP1 | −0.699573723 | 0.001565532 |
| PTPN6 | −0.700270084 | 0.030333328 |
| CAMKK1 | −0.701306 | 0.000548552 |
| LSP1 | −0.705871079 | 4.94E−05 |
| TGFA | −0.707138876 | 4.25E−07 |
| POLR2F | −0.71055219 | 0.00039211 |
| CPPED1 | −0.710588323 | 0.008003467 |
| EREG | −0.710717174 | 0.017619415 |
| TOP2B | −0.713729191 | 0.039021131 |
| TNFSF13 | −0.714436948 | 1.50E−12 |
| COL2A1 | −0.724419429 | 0.000217458 |
| IL16 | −0.725531548 | 8.89E−06 |
| SPARC | −0.727160498 | 0.000224126 |
| TDP1 | −0.731546845 | 0.001273238 |
| ILKAP | −0.734156886 | 0.002824572 |
| CLEC2L | −0.735337845 | 1.08E−05 |
| PIKFYVE | −0.737186452 | 0.003712195 |
| SMPD3 | −0.73793661 | 0.003582148 |
| CD22 | −0.747897388 | 1.67E−07 |
| FCN1 | −0.749294965 | 2.37E−11 |
| SWAP70 | −0.750557173 | 1.06E−05 |
| ARG1 | −0.758422452 | 0.000129364 |
| TIMP3 | −0.75868893 | 0.022568769 |
| VIM | −0.767926037 | 0.001092979 |
| BAG4 | −0.768092266 | 0.016422612 |
| PQBP1 | −0.775987743 | 0.001393261 |
| STX5 | −0.777858387 | 0.023661849 |
| GIMAP8 | −0.779515267 | 0.002902732 |
| FABP5 | −0.789508834 | 0.005440735 |
| ARHGAP30 | −0.792172071 | 0.000863559 |
| RPL14 | −0.794988016 | 2.23E−05 |
| PDGFA | −0.805127209 | 0.000237792 |
| RPE | −0.821339301 | 0.036064717 |
| SF3B4 | −0.826444767 | 0.004847066 |
| CRHR1 | −0.833388292 | 0.045099365 |
| PDE4D | −0.836027855 | 2.79E−05 |
| EIF1AX | −0.840456894 | 0.019154254 |
| CAMLG | −0.84110768 | 0.003857605 |
| PCARE | −0.847683597 | 0.003889971 |
| HSBP1 | −0.855488642 | 0.001328335 |
| GADD45B | −0.855745252 | 0.002961018 |
| CYB5R2 | −0.860817665 | 0.000132349 |
| TIGAR | −0.867795097 | 0.000125506 |
| TNFAIP8 | −0.871893486 | 0.000418948 |
| ELOA | −0.874226635 | 0.000828517 |
| PFKFB2 | −0.879769458 | 0.004297361 |
| CEBPB | −0.894356306 | 1.38E−06 |
| CD40LG | −0.900264289 | 0.001004797 |
| STAU1 | −0.902193358 | 0.002696648 |
| COL9A2 | −0.916607811 | 0.00425954 |
| PDGFB | −0.929167811 | 0.000447415 |
| HBEGF | −0.936170494 | 4.11E−05 |
| PPBP | −0.938264974 | 0.000642031 |
| ANXA2 | −0.964716146 | 0.006457981 |
| AIF1 | −0.966967183 | 0.012371769 |
| SDC4 | −0.968325075 | 2.16E−09 |
| ANXA4 | −0.969055871 | 0.001240998 |
| EIF2AK2 | −0.976282358 | 0.04297901 |
| OSM | −0.978323176 | 2.38E−06 |
| NPHS2 | −0.982133887 | 0.002408618 |
| MMP9 | −0.986192931 | 3.50E−09 |
| SERPINB8 | −0.990143425 | 9.65E−05 |
| FUS | −1.002722282 | 1.12E−07 |
| IL18RAP | −1.002815156 | 0.000567852 |
| ANGPT1 | −1.004114706 | 1.13E−06 |
| CAPG | −1.004306596 | 2.70E−06 |
| S100P | −1.029289266 | 5.40E−06 |
| PF4 | −1.030215489 | 0.000155044 |
| CASP8 | −1.036831402 | 0.00043543 |
| AK2 | −1.03716148 | 0.00369652 |
| SRP14 | −1.043914834 | 2.45E−05 |
| IL1RN | −1.04504713 | 6.57E−07 |
| TNFAIP8L2 | −1.048515632 | 0.022029758 |
| NADK | −1.050215028 | 6.42E−06 |
| SERPINB1 | −1.057116367 | 0.004224636 |
| NAMPT | −1.057605782 | 0.007426807 |
| FEN1 | −1.060100624 | 0.000287093 |
| IL18 | −1.066486672 | 1.52E−06 |
| HK2 | −1.068204488 | 0.004344286 |
| GMFG | −1.075374489 | 0.00039211 |
| PRDX5 | −1.077626804 | 1.02E−05 |
| ANXA1 | −1.090434807 | 1.15E−07 |
| PADI4 | −1.094933493 | 0.002913206 |
| OLR1 | −1.142339913 | 1.65E−08 |
| BDNF | −1.197037596 | 1.03E−06 |
| TNFSF14 | −1.248354779 | 3.78E−09 |
| ZBP1 | −1.27348416 | 1.08E−05 |
| PARP1 | −1.275862704 | 1.06E−05 |
| EDF1 | −1.317296348 | 0.000254073 |
| IPCEF1 | −1.322307211 | 0.00039211 |
| DOC2B | −1.324347407 | 0.000155044 |
| TMSB10 | −1.337382098 | 6.57E−07 |
| APBB1IP | −1.340268231 | 4.26E−09 |
| MMP8 | −1.357563592 | 2.93E−06 |
| CASP10 | −1.371061093 | 5.64E−05 |
| PXN | −1.371966177 | 2.23E−08 |
| APEX1 | −1.379532894 | 6.05E−08 |
| HCLS1 | −1.408164186 | 6.55E−10 |
| IL1B | −1.476286521 | 2.88E−05 |
| IDO1 | −1.519712742 | 3.08E−08 |
| MNDA | −1.54299201 | 2.38E−06 |
| EGLN1 | −1.54478424 | 5.00E−06 |
| RASSF2 | −1.550118858 | 0.000157416 |
| S100A11 | −1.558599009 | 6.49E−13 |
| IDO1 | −1.560668089 | 3.57E−09 |
| LRCH4 | −1.56730594 | 2.72E−06 |
| IDO1 | −1.594677889 | 7.12E−08 |
| S100A12 | −1.611140523 | 1.65E−08 |
| ANXA3 | −1.649435143 | 1.47E−08 |
| IDO1 | −1.692209182 | 1.25E−08 |
| SMNDC1 | −1.701570115 | 6.63E−07 |
| NEDD9 | −1.721181829 | 2.82E−05 |
| TOR1AIP1 | −1.735706926 | 1.92E−07 |
| PPP1R12B | −1.799865688 | 0.001453201 |
| RBP7 | −1.860879347 | 3.17E−15 |
| RAB44 | −1.877800867 | 3.81E−09 |
| CLC | −1.887308789 | 4.62E−11 |
| BCL2L15 | −1.940395293 | 4.30E−12 |
| DAPK2 | −2.227981149 | 5.06E−10 |
| NCF2 | −2.342476207 | 2.37E−11 |
| CASP1 | −2.445582232 | 1.29E−06 |

TABLE 3
Genes With Significant Alteration in Expression
in NDMM Compared to Healthy Subjects

| Gene | NDMM.vs.Normal_logFC | P value |
|---|---|---|
| FCRLB | 3.390039085 | 1.63E−19 |
| TNFRSF13B | 2.587959174 | 5.53E−32 |
| SLAMF7 | 2.584976266 | 2.45E−25 |
| FCRL5 | 2.355547536 | 2.34E−22 |
| TNFRSF17 | 2.108616814 | 2.57E−48 |
| SLAMF1 | 1.946086509 | 1.32E−08 |
| IL5RA | 1.905800337 | 6.36E−10 |
| MZB1 | 1.859917165 | 1.71E−20 |
| CNTN5 | 1.850539122 | 6.49E−19 |
| EPO | 1.703022556 | 1.29E−08 |
| CD79B | 1.669549079 | 1.33E−13 |
| SERPINH1 | 1.553451669 | 0.022803287 |
| KRT8 | 1.509699489 | 0.000772969 |
| TXNDC5 | 1.506694355 | 6.42E−08 |
| KIF22 | 1.426596378 | 0.000207153 |
| MICB_MICA | 1.360534995 | 0.013007132 |
| SPINT3 | 1.350414906 | 0.048657796 |
| AKR7L | 1.344603308 | 3.69E−07 |
| ZNRD2 | 1.344103225 | 1.01E−05 |
| FUOM | 1.259802248 | 5.12E−05 |
| ICAM3 | 1.246200654 | 8.11E−22 |
| CRELD2 | 1.212412646 | 3.69E−07 |
| ADGRG1 | 1.157895419 | 2.61E−05 |
| CEACAM16 | 1.087156611 | 0.000127955 |
| IL6 | 1.06353339 | 0.000657249 |
| CSF3 | 1.036696446 | 4.53E−07 |
| CHGA | 1.028378638 | 0.000735735 |
| SDC1 | 1.019576452 | 2.81E−08 |
| THBS2 | 1.006657826 | 2.37E−07 |
| DUT | 0.998144178 | 0.015076992 |
| ACADSB | 0.986868396 | 0.011175724 |
| QPCT | 0.981304044 | 3.30E−12 |
| MYDGF | 0.976712916 | 0.030900034 |
| ITM2A | 0.970819876 | 5.95E−07 |
| AIFM1 | 0.956533884 | 0.041061108 |
| PRDX1 | 0.956512844 | 0.016635198 |
| NDUFB7 | 0.951034453 | 0.012160311 |
| MILR1 | 0.94185444 | 3.18E−06 |
| MMUT | 0.93037544 | 0.014443851 |
| ADA2 | 0.916937258 | 3.18E−12 |
| RLN2 | 0.906730793 | 0.000485727 |
| FCRL2 | 0.894211459 | 0.001085982 |
| CD48 | 0.889908139 | 1.21E−13 |
| EIF2AK3 | 0.883174413 | 0.01627819 |
| HAO1 | 0.881297953 | 0.02416283 |
| IL6 | 0.870813047 | 0.002166218 |
| RALB | 0.859093698 | 0.001556887 |
| CLPP | 0.834759927 | 0.049235561 |
| TOMM20 | 0.822138213 | 0.005267589 |
| ECHS1 | 0.820955378 | 0.049495795 |
| RARRES1 | 0.816945622 | 0.001096172 |
| GAST | 0.815356696 | 0.047475389 |
| CD274 | 0.812946084 | 0.000461744 |
| PNMA1 | 0.799621502 | 0.027220795 |
| FTCD | 0.793608835 | 0.000778743 |
| LY9 | 0.786294328 | 3.55E−06 |
| NDUFS6 | 0.784120541 | 0.014310997 |
| TDO2 | 0.781573472 | 0.014443851 |
| ADH1B | 0.76671181 | 0.000666512 |
| IL6 | 0.744862436 | 0.009122181 |
| PDZK1 | 0.734551973 | 0.011855861 |
| RLN1 | 0.732709152 | 0.011290086 |
| IL6 | 0.730036824 | 0.014909289 |
| MDK | 0.728318751 | 0.000542625 |
| LAG3 | 0.712068077 | 6.09E−05 |
| TEX101 | 0.706920563 | 0.033963812 |
| KHK | 0.70093205 | 0.011175724 |
| PDIA4 | 0.697507503 | 0.044663563 |
| CEACAM19 | 0.683481717 | 0.000375517 |
| ST6GAL1 | 0.681779446 | 7.06E−07 |
| B2M | 0.677832077 | 9.26E−10 |
| EFEMP1 | 0.67565016 | 0.000262874 |
| SSBP1 | 0.673202982 | 0.008147255 |
| NCR3LG1 | 0.667424363 | 1.62E−06 |
| GALNT3 | 0.662951373 | 0.000137104 |
| LY96 | 0.636649559 | 0.001810499 |
| IMMT | 0.628110383 | 0.002845122 |
| IGFBP2 | 0.625436069 | 0.006604819 |
| RNASET2 | 0.62352801 | 7.04E−08 |
| TP53 | 0.611402393 | 0.048453002 |
| CKB | 0.607149578 | 0.00578242 |
| SULT2A1 | 0.602122289 | 0.004593839 |
| GDF15 | 0.601884614 | 4.48E−05 |
| GPRC5C | 0.594234296 | 0.014443851 |
| KLK13 | 0.594112645 | 0.006815177 |
| ATRAID | 0.593935085 | 4.15E−07 |
| IL17C | 0.588503435 | 0.015811808 |
| KRT18 | 0.588098668 | 0.049163142 |
| XCL1 | 0.580597167 | 0.014909289 |
| MRPL58 | 0.574737535 | 0.040599462 |
| VWF | 0.57115521 | 0.009223196 |
| VSIG2 | 0.558108879 | 0.016635198 |
| NOS2 | 0.556236785 | 0.018255842 |
| CAPS | 0.552613918 | 0.004622367 |
| GSAP | 0.549718028 | 0.019670949 |
| AGXT | 0.548179676 | 0.023974517 |
| SRPX | 0.547003295 | 0.001759652 |
| WFDC2 | 0.542416125 | 0.000737827 |
| SEL1L | 0.54213755 | 0.000137104 |
| ADAMTS1 | 0.540057923 | 0.000657249 |
| CBS | 0.535388828 | 0.017026898 |
| FBLN2 | 0.534728785 | 2.78E−05 |
| ARTN | 0.531763085 | 0.013805556 |
| RAB10 | 0.528356648 | 0.041993219 |
| LILRB4 | 0.523417326 | 0.005016449 |
| MYO6 | 0.519283124 | 0.048851771 |
| CES2 | 0.51621798 | 0.039833078 |
| SLAMF6 | 0.513623018 | 0.0047232 |
| COCH | 0.512676452 | 0.000148752 |
| NTF4 | 0.508114624 | 0.001810499 |
| ELN | 0.505998881 | 0.004671612 |
| PPL | 0.505731535 | 0.004158206 |
| CDCP1 | 0.502393931 | 0.023939125 |
| PTS | 0.497291265 | 0.044945229 |
| IL22 | 0.494969405 | 0.021431827 |
| ANXA10 | 0.493401234 | 0.040029942 |
| LAMP3 | 0.488971908 | 0.029138941 |
| PGA4 | 0.482734608 | 0.020339109 |
| PROK1 | 0.482714415 | 0.044586244 |
| FGL1 | 0.47985435 | 0.008659364 |
| FGA | 0.478232424 | 0.010589912 |
| IFNLR1 | 0.467308761 | 0.014105556 |
| PMCH | 0.453445804 | 0.044324362 |
| TLR1 | 0.450567522 | 0.00604946 |
| NPTX2 | 0.448366321 | 7.14E−05 |
| LTBP2 | 0.437436678 | 0.001915445 |
| LTBP3 | 0.434311881 | 0.009975352 |
| REG3A | 0.43364282 | 0.04626337 |
| PLA2G15 | 0.432116597 | 6.73E−05 |
| DCXR | 0.430441386 | 0.029205745 |
| LRRC37A2 | 0.430182849 | 0.011093082 |
| EDA2R | 0.423429095 | 0.047700463 |
| CD86 | 0.420437707 | 0.003121694 |
| PCDH17 | 0.415999244 | 0.002734765 |
| CYTL1 | 0.41318279 | 0.002545763 |
| NT5C1A | 0.412321578 | 0.029558312 |
| ACE2 | 0.411670927 | 0.017955442 |
| TNFSF8 | 0.410784871 | 0.000737827 |
| YAP1 | 0.410683382 | 0.005614964 |
| IL12RB1 | 0.409496362 | 0.010666522 |
| ANGPTL2 | 0.401841838 | 0.008199654 |
| C1QTNF5 | 0.401158938 | 0.00015079 |
| TINAGL1 | 0.399924411 | 1.03E−06 |
| RNASE10 | 0.398610572 | 0.027761467 |
| OCLN | 0.395219652 | 0.025206382 |
| ITGBL1 | 0.395002454 | 0.001973218 |
| SIGLEC1 | 0.393677883 | 0.008568461 |
| MYOC | 0.391824289 | 0.031853528 |
| GIPC2 | 0.390642018 | 0.008582657 |
| ASAH1 | 0.389574394 | 0.042599609 |
| RBFOX3 | 0.388926561 | 0.026318563 |
| GAS6 | 0.386258594 | 0.000202869 |
| NHLRC3 | 0.382874203 | 0.000693173 |
| CHGB | 0.38162971 | 0.013003669 |
| ICAM2 | 0.380651107 | 0.000154934 |
| TPP1 | 0.378061161 | 0.014467717 |
| WNT9A | 0.376528917 | 0.008172637 |
| ACP5 | 0.373404605 | 0.000666512 |
| RNASE6 | 0.370622717 | 0.000657249 |
| MAN1A2 | 0.363381906 | 0.000120596 |
| BTN3A2 | 0.361238993 | 0.031321885 |
| PRSS22 | 0.359048645 | 0.028459448 |
| EDNRB | 0.358135383 | 0.039466771 |
| CTHRC1 | 0.356006778 | 0.001306608 |
| PDIA3 | 0.352885058 | 0.026759673 |
| NPC2 | 0.352296628 | 0.00147903 |
| NRCAM | 0.34960564 | 0.001810499 |
| SPINK2 | 0.349410525 | 0.008582657 |
| IFI30 | 0.349359272 | 0.044663563 |
| VEGFB | 0.348810368 | 0.039826277 |
| HS6ST2 | 0.348745386 | 0.02456424 |
| C7 | 0.344752468 | 0.000108396 |
| MRC1 | 0.344092238 | 0.000181585 |
| PIGR | 0.33788363 | 0.011175724 |
| TIMP1 | 0.335474003 | 0.001759652 |
| FN1 | 0.334807794 | 0.033594091 |
| CCN5 | 0.334639832 | 0.044002021 |
| EFNA1 | 0.325659101 | 0.033594091 |
| CKAP4 | 0.324671823 | 0.029138941 |
| S100A13 | 0.32414156 | 0.033875199 |
| SPON2 | 0.323242913 | 0.020997527 |
| RELT | 0.322762552 | 0.007909433 |
| TMPRSS11D | 0.320357135 | 0.019502125 |
| COL4A1 | 0.319983053 | 0.027761467 |
| GM2A | 0.30893665 | 0.02490334 |
| PSAP | 0.306188466 | 0.002846482 |
| LGMN | 0.304878129 | 0.012182696 |
| ANGPTL1 | 0.304573763 | 0.001810499 |
| ADAMTSL4 | 0.301845703 | 0.00750737 |
| LRIG3 | 0.298641755 | 0.023921033 |
| ADGRE2 | 0.297491053 | 0.004618567 |
| ADGRE5 | 0.29720826 | 0.008148609 |
| SCRG1 | 0.294649829 | 0.035708695 |
| CD74 | 0.292612358 | 0.03674457 |
| CPXM2 | 0.288793997 | 0.01442445 |
| IL10RB | 0.287607821 | 0.012122962 |
| SHISA5 | 0.286629004 | 0.022803287 |
| RNASE1 | 0.285462865 | 0.026318563 |
| RNASE4 | 0.284100584 | 0.002734765 |
| PAM | 0.269145825 | 0.006800391 |
| IGFBP7 | 0.26776318 | 0.006690624 |
| MFAP4 | 0.262474072 | 0.018513735 |
| BMPER | 0.259946234 | 0.000376291 |
| DIPK2B | 0.254385836 | 0.014370162 |
| NRP2 | 0.246350681 | 0.043593279 |
| NOMO1 | 0.243671198 | 0.003279778 |
| CRIM1 | 0.239988127 | 0.002138764 |
| EGFLAM | 0.237159443 | 0.047239866 |
| EDN1 | 0.235681429 | 0.03502372 |
| INSR | 0.218679963 | 0.00134009 |
| TGFBI | 0.213081714 | 0.043593279 |
| ERBB4 | 0.211182132 | 0.012686149 |
| SERPINC1 | −0.122423069 | 0.003418201 |
| F9 | −0.136812271 | 0.044663563 |
| ENG | −0.137511857 | 0.039229341 |
| MET | −0.14370667 | 0.044002021 |
| SERPINF2 | −0.154663954 | 0.008511384 |
| SERPING1 | −0.155340605 | 0.004027574 |
| C1R | −0.168908991 | 0.027761467 |
| IL1R2 | −0.16893358 | 0.017127991 |
| CA4 | −0.170438256 | 0.046925422 |
| MENT | −0.179451969 | 0.014553228 |
| F10 | −0.181376483 | 0.008511384 |
| RECK | −0.188486206 | 0.010103469 |
| PEAR1 | −0.189805802 | 0.044506458 |
| LCAT | −0.19095447 | 0.005016449 |
| TTR | −0.191444886 | 0.014101358 |
| CPB2 | −0.194739515 | 0.00582751 |
| TYRO3 | −0.197370491 | 0.020271056 |
| CD99 | −0.200746389 | 0.029884886 |
| ITGB1 | −0.202786055 | 0.003923973 |
| IL13RA1 | −0.204487436 | 0.005627393 |
| ATRN | −0.204827827 | 0.000214606 |
| PROC | −0.207374048 | 0.020955791 |
| AFM | −0.207660958 | 0.002890604 |
| C2 | −0.21033371 | 0.002183262 |
| FAM171B | −0.212151092 | 0.022765771 |
| A1BG | −0.213355019 | 0.000181585 |
| ACAN | −0.214051155 | 0.018383119 |
| LYPD3 | −0.217354297 | 0.028971873 |
| NCAM2 | −0.217667737 | 0.013313533 |
| IGDCC4 | −0.221533938 | 0.007011818 |
| CNTN4 | −0.224192504 | 0.004907949 |
| CD58 | −0.224894215 | 0.000375517 |
| BCHE | −0.225015725 | 0.002311845 |
| HEPH | −0.22563875 | 0.003446019 |
| ADGRD1 | −0.227473715 | 0.038210677 |
| PI16 | −0.236864102 | 0.02416283 |
| TF | −0.236866351 | 0.000371573 |
| KIT | −0.237594355 | 0.011855861 |
| TFPI | −0.237676022 | 0.009554936 |
| MANSC1 | −0.238592562 | 0.031321885 |
| TNFRSF21 | −0.239556039 | 0.013879004 |
| ORM1 | −0.240334745 | 0.007909433 |
| ROBO2 | −0.241657472 | 0.00170135 |
| APOA4 | −0.242263462 | 0.030338333 |
| SERPINA5 | −0.244659439 | 0.01627819 |
| LAMP2 | −0.245304158 | 0.000736715 |
| GKN1 | −0.245422351 | 0.044518273 |
| APOA1 | −0.247441346 | 0.000254891 |
| CLU | −0.25046918 | 0.000120344 |
| CNTN3 | −0.25423724 | 0.031243755 |
| KDR | −0.254259676 | 0.003449435 |
| CDHR5 | −0.256642101 | 0.027761467 |
| ERBB2 | −0.258026296 | 0.000150391 |
| CD209 | −0.258043209 | 0.029261311 |
| AHNAK2 | −0.261659208 | 0.044663563 |
| CD99L2 | −0.26690055 | 0.002206391 |
| CRLF1 | −0.270393948 | 0.001556887 |
| PLA2G7 | −0.270593207 | 0.008568461 |
| PTPRC | −0.270996977 | 0.004318311 |
| GRIK2 | −0.271199687 | 0.048685345 |
| F12 | −0.272414605 | 0.039272131 |
| BOC | −0.274265618 | 0.036799167 |
| VCAN | −0.27484417 | 0.018139285 |
| SERPINA6 | −0.277234252 | 6.13E−05 |
| PRSS53 | −0.278468087 | 0.044663563 |
| PDGFC | −0.28087679 | 0.015726228 |
| ERBB3 | −0.281567796 | 7.12E−06 |
| CR1 | −0.284984193 | 0.00907211 |
| DNER | −0.286708547 | 0.000430556 |
| IL31RA | −0.289876466 | 0.044002021 |
| CHL1 | −0.298440752 | 0.000681755 |
| FAP | −0.301266317 | 0.000120228 |
| ITGA2 | −0.302158 | 0.01566977 |
| KIRREL2 | −0.302713441 | 0.042573804 |
| CLEC1A | −0.302754345 | 0.036799167 |
| ENPP6 | −0.304240231 | 0.020930086 |
| ADCYAP1R1 | −0.304569513 | 0.022803287 |
| F11 | −0.316117529 | 0.009834094 |
| DPP4 | −0.316684791 | 0.000154934 |
| LILRA5 | −0.318521925 | 0.00275005 |
| TCN1 | −0.321819627 | 0.004390798 |
| SDK2 | −0.323271921 | 0.008846007 |
| SMAD5 | −0.325201897 | 0.013567868 |
| CEACAM6 | −0.327570767 | 0.028442601 |
| LTB | −0.329686905 | 0.011855861 |
| GDF2 | −0.331965494 | 0.004718843 |
| EGFR | −0.333129922 | 1.55E−09 |
| KLKB1 | −0.334262365 | 5.78E−06 |
| CPOX | −0.3354966 | 0.003923973 |
| IGF1R | −0.338118178 | 0.015794505 |
| CD1C | −0.339053813 | 0.000270566 |
| MCAM | −0.343594 | 0.003973929 |
| GP1BA | −0.348235408 | 0.003666146 |
| CNGB3 | −0.352248801 | 0.012727781 |
| ITGAV | −0.356247812 | 0.009975352 |
| CD101 | −0.358352175 | 0.02837536 |
| PLXDC2 | −0.35947032 | 0.001144616 |
| IMPG1 | −0.370326193 | 0.001329181 |
| APOM | −0.371648636 | 0.000112838 |
| CD244 | −0.372572946 | 0.023528068 |
| MED21 | −0.374377593 | 0.0047232 |
| ICAM5 | −0.375054887 | 0.011290086 |
| TPSD1 | −0.377717189 | 0.033963812 |
| CDH23 | −0.382658644 | 0.011405603 |
| MEGF9 | −0.383722848 | 4.10E−09 |
| FGFBP2 | −0.385114114 | 0.005242316 |
| CTSV | −0.388859465 | 0.040431648 |
| GHR | −0.389063206 | 0.000195844 |
| CCN4 | −0.392032499 | 0.022664638 |
| LEPR | −0.392524703 | 0.000120596 |
| CD84 | −0.394597261 | 0.028971873 |
| PRG3 | −0.400491498 | 0.022419823 |
| IGFBP3 | −0.402626353 | 4.48E−05 |
| ITGB2 | −0.40525072 | 0.000268101 |
| HMOX1 | −0.406084281 | 0.010373137 |
| LGALS3 | −0.407275944 | 0.009147954 |
| BST1 | −0.407290616 | 9.89E−05 |
| ITGAM | −0.409371583 | 0.003314091 |
| TLR3 | −0.41223877 | 0.02985327 |
| SNED1 | −0.4137644 | 0.00582036 |
| FGFR4 | −0.414904851 | 0.015904436 |
| GFRAL | −0.41990924 | 0.013065108 |
| F3 | −0.420603533 | 0.027595666 |
| CDON | −0.422860532 | 3.69E−07 |
| PRTG | −0.423519216 | 0.008568461 |
| AKAP12 | −0.424851443 | 1.79E−06 |
| GIMAP7 | −0.429084593 | 0.026585085 |
| ITGA11 | −0.43289787 | 0.003082781 |
| CRISP3 | −0.438372449 | 1.58E−09 |
| ADAM8 | −0.440809053 | 0.002752363 |
| EVPL | −0.441314678 | 0.044663563 |
| CD34 | −0.441820003 | 1.29E−08 |
| PCDH1 | −0.445024201 | 0.000116172 |
| ADAM23 | −0.446159283 | 0.029905942 |
| SOWAHA | −0.447089567 | 0.020693781 |
| C1S | −0.450564318 | 6.95E−08 |
| WFIKKN1 | −0.450792277 | 0.002992861 |
| WFDC1 | −0.452164545 | 0.000274251 |
| ADGRG2 | −0.452364039 | 3.26E−06 |
| ICAM4 | −0.452698826 | 0.023740752 |
| BAG3 | −0.455422283 | 0.001251234 |
| TGFA | −0.455910499 | 0.025504061 |
| GPC5 | −0.458226184 | 0.028338097 |
| SIGLEC9 | −0.46442927 | 0.007103525 |
| AHNAK | −0.465072807 | 0.009591098 |
| MPO | −0.466134907 | 0.025451142 |
| S100A14 | −0.470484538 | 0.015196898 |
| DPEP2 | −0.47355554 | 0.005451703 |
| SELL | −0.474417755 | 3.43E−09 |
| KLRC1 | −0.47501598 | 0.045797807 |
| CR2 | −0.479725546 | 0.001043993 |
| KLRB1 | −0.48363648 | 0.004593839 |
| DHPS | −0.487759791 | 0.018383119 |
| NCS1 | −0.489245783 | 0.009122181 |
| IFT20 | −0.496402879 | 0.044945229 |
| TNFRSF10C | −0.500431641 | 0.000250671 |
| NEK7 | −0.502842727 | 0.040290913 |
| CLMP | −0.505142363 | 0.004618567 |
| VEGFC | −0.505856507 | 0.008582657 |
| CNDP1 | −0.506282055 | 0.000164499 |
| KEL | −0.50871508 | 0.000717471 |
| TPPP3 | −0.509424754 | 0.006800391 |
| SWAP70 | −0.510626563 | 0.039889336 |
| TNFSF12 | −0.511549641 | 0.003297323 |
| BPIFB2 | −0.520321097 | 0.040029942 |
| AFP | −0.52364649 | 0.01252306 |
| VSTM1 | −0.52991213 | 0.018332701 |
| NMRK2 | −0.53154867 | 0.001463427 |
| GAGE2A | −0.53195436 | 0.043865111 |
| BCL7B | −0.533639968 | 0.010373137 |
| CD5 | −0.533646765 | 0.008582657 |
| CNPY2 | −0.535288905 | 0.041219617 |
| CLEC4A | −0.53545737 | 0.000814996 |
| CD300E | −0.538217337 | 0.008582657 |
| SAT2 | −0.541839847 | 0.040606416 |
| FCN2 | −0.545337594 | 0.000601228 |
| PDCL2 | −0.546370753 | 0.016635631 |
| CDA | −0.549855768 | 0.011093082 |
| HNMT | −0.549903419 | 0.028459448 |
| CEACAM8 | −0.551079704 | 0.019456621 |
| RET | −0.556147844 | 0.004178195 |
| GLRX | −0.572460497 | 0.038913006 |
| RPL14 | −0.576960836 | 0.032371539 |
| MORF4L2 | −0.577987286 | 0.008659364 |
| APOL1 | −0.582973959 | 0.000567204 |
| CEP164 | −0.584123004 | 0.039833078 |
| CCN2 | −0.594946136 | 0.0023978 |
| GPD1 | −0.601427645 | 0.000756332 |
| NGFR | −0.608267943 | 0.000371573 |
| GRK5 | −0.609350285 | 0.032939162 |
| CLEC4D | −0.610389508 | 0.016966543 |
| IL31 | −0.610682723 | 0.021503348 |
| PZP | −0.611179465 | 0.02948792 |
| CBLN4 | −0.613112903 | 1.06E−05 |
| RNF43 | −0.613213643 | 0.001759652 |
| ODAM | −0.616390462 | 0.008659364 |
| APP | −0.616678598 | 0.011175724 |
| NAPRT | −0.620913456 | 0.014747682 |
| IL16 | −0.622265047 | 0.006800391 |
| MMP1 | −0.627378965 | 0.039624532 |
| SMPD3 | −0.633972518 | 0.044663563 |
| ARG1 | −0.635920666 | 0.021503348 |
| XRCC4 | −0.636675737 | 0.0068514 |
| CEBPB | −0.647392709 | 0.014721299 |
| COL2A1 | −0.658019035 | 0.01448057 |
| SPRING1 | −0.665526686 | 0.000329924 |
| CSF2RA | −0.666201217 | 0.000262874 |
| DEFB116 | −0.668840363 | 0.001561388 |
| ITGA6 | −0.688715966 | 0.004027574 |
| TNFAIP8 | −0.692586599 | 0.026939636 |
| POLR2F | −0.693242921 | 0.011093082 |
| FUS | −0.694775995 | 0.010744125 |
| ILKAP | −0.708481291 | 0.033258633 |
| CLEC5A | −0.709197587 | 2.00E−07 |
| TNFSF13B | −0.719333708 | 1.04E−10 |
| AZU1 | −0.721662386 | 0.036359285 |
| SERPINB8 | −0.742345615 | 0.040592138 |
| PDGFA | −0.770794201 | 0.01019299 |
| OSM | −0.773450375 | 0.008659364 |
| MFAP3 | −0.781158588 | 0.006052764 |
| CD40LG | −0.78593209 | 0.038329466 |
| FASLG | −0.788138019 | 3.69E−07 |
| SPARC | −0.804378637 | 0.002369212 |
| STAU1 | −0.808572249 | 0.028971873 |
| CLEC2L | −0.818001172 | 0.000303569 |
| CLEC4C | −0.818093667 | 3.18E−06 |
| ELOA | −0.820797513 | 0.021503348 |
| CAPG | −0.834010926 | 0.0058238 |
| CD6 | −0.836881987 | 1.61E−05 |
| PDE4D | −0.858221877 | 0.000485727 |
| NADK | −0.859951673 | 0.008988722 |
| PCARE | −0.870468608 | 0.014338209 |
| PPBP | −0.878490537 | 0.019579545 |
| PXN | −0.880788884 | 0.013567868 |
| SRP14 | −0.882241174 | 0.011175724 |
| PDGFB | −0.88341408 | 0.014443851 |
| APBB1IP | −0.896191776 | 0.00664328 |
| PRDX5 | −0.898071172 | 0.00907211 |
| ANXA1 | −0.911777428 | 0.001659422 |
| ANGPT1 | −0.916386577 | 0.001416452 |
| DOC2B | −0.916505162 | 0.044045469 |
| VIM | −0.917841252 | 0.00312446 |
| FCN1 | −0.931945274 | 2.04E−10 |
| CAMLG | −0.980575293 | 0.010690558 |
| TP73 | −0.983184604 | 0.02837536 |
| IL18RAP | −1.004333556 | 0.010993778 |
| IL1RN | −1.009958373 | 0.000485727 |
| PARP1 | −1.018469394 | 0.013065108 |
| PF4 | −1.025586726 | 0.00582751 |
| APEX1 | −1.027965705 | 0.004682944 |
| HBEGF | −1.028096345 | 0.000836502 |
| CD22 | −1.030457507 | 3.63E−08 |
| MMP9 | −1.04778443 | 2.72E−06 |
| IPCEF1 | −1.062182753 | 0.043224987 |
| IL18 | −1.069135571 | 0.000451464 |
| IDO1 | −1.092245783 | 0.003923973 |
| S100A12 | −1.108943094 | 0.007103525 |
| RNASE3 | −1.116232271 | 0.021688976 |
| TNFSF14 | −1.150774805 | 8.63E−05 |
| TOR1AIP1 | −1.151928812 | 0.017068076 |
| NEDD9 | −1.172384415 | 0.02899556 |
| IDO1 | −1.174722071 | 0.002620609 |
| TNFSF13 | −1.175409707 | 1.34E−19 |
| SMNDC1 | −1.176478844 | 0.017022891 |
| HCLS1 | −1.179558139 | 0.000188059 |
| TMSB10 | −1.180882981 | 0.001688315 |
| SDC4 | −1.211995307 | 1.23E−08 |
| BDNF | −1.226109156 | 0.000254891 |
| IDO1 | −1.242772982 | 0.003104477 |
| IDO1 | −1.244611573 | 0.003295343 |
| BCL2L15 | −1.359432149 | 0.000555854 |
| OLR1 | −1.391400998 | 2.18E−07 |
| RAB44 | −1.400411894 | 0.001967217 |
| ANXA3 | −1.402129193 | 0.000561628 |
| DAPK2 | −1.425978534 | 0.005651962 |
| S100A11 | −1.52266887 | 1.35E−07 |
| MMP8 | −1.575107642 | 6.13E−05 |
| RBP7 | −1.608210195 | 3.69E−07 |
| MNDA | −1.611988463 | 0.000303569 |
| CLC | −1.70629978 | 1.19E−05 |
| NCF2 | −1.734198053 | 0.000407791 |
| CASP1 | −1.999416682 | 0.001860632 |

TABLE 4
Genes With Significant Alteration in Expression in Low-risk
SMM Compared to High-risk SMM Subjects

| Gene | Fold Change in Expression | P Value |
|---|---|---|
| TNFRSF17 | −0.996676127 | 1.30E−15 |
| TNFRSF13B | −1.221668932 | 3.42E−11 |
| CNTN5 | −1.107564113 | 1.55E−08 |
| FCRL5 | −1.109396958 | 1.18E−07 |
| TNFSF13 | 0.586041994 | 1.54E−07 |
| ICAM3 | −0.642433711 | 3.84E−07 |
| CD5L | 0.774284255 | 6.26E−06 |
| SLAMF7 | −0.990392449 | 8.21E−06 |
| CD79B | −0.942648856 | 9.28E−06 |
| MZB1 | −0.694306174 | 0.000103 |
| IL5RA | −1.125158383 | 0.000108 |
| SEPTIN3 | 0.788527093 | 0.000171 |

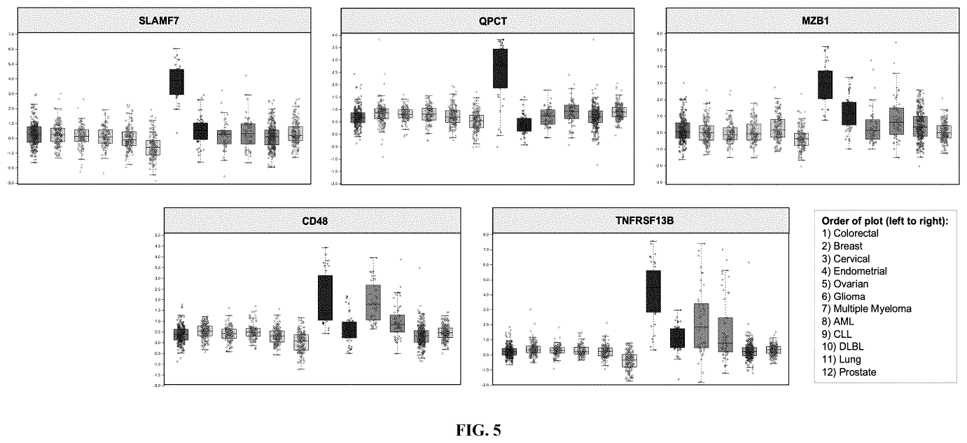

Consistent with previous findings, baseline BCMA levels was also significantly elevated in SMM-MM progressors vs. SMM non-progressors, further supporting the potential utility of BCMA measurements during routine blood tests of precursor MM patients. Proinflammatory cytokines were also identified, including IL1, IL5, IL6, IL16, and IL18, known to create a bone marrow (BM) environment that promotes malignant cell development by suppressing the microenvironment, promoting cellular adhesion, or increasing angiogenesis. Disease signals specific to MM were also identified (FIG. 5).

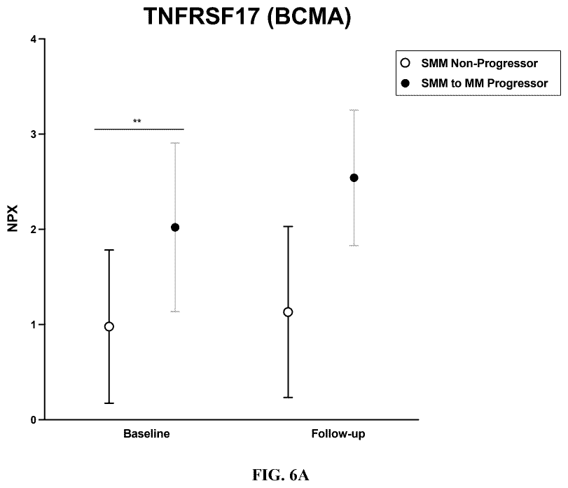

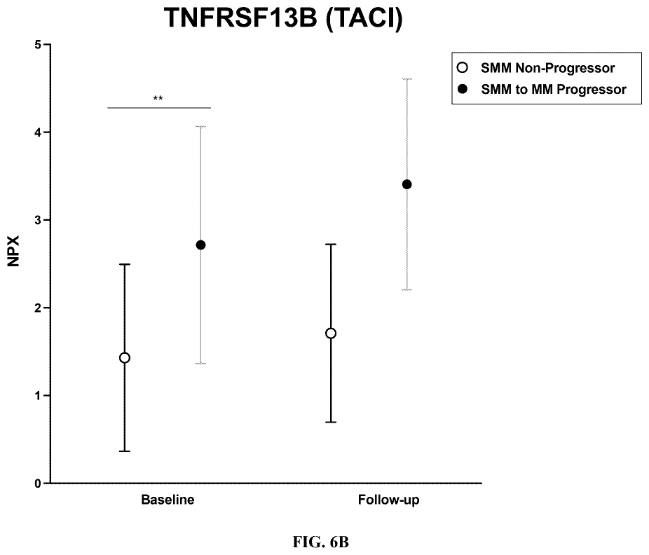

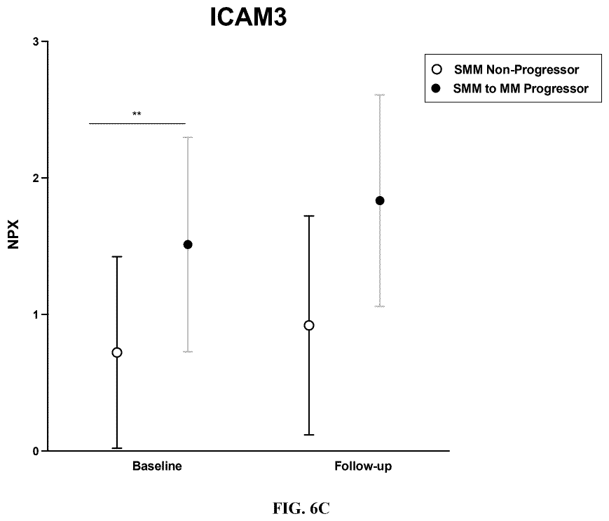

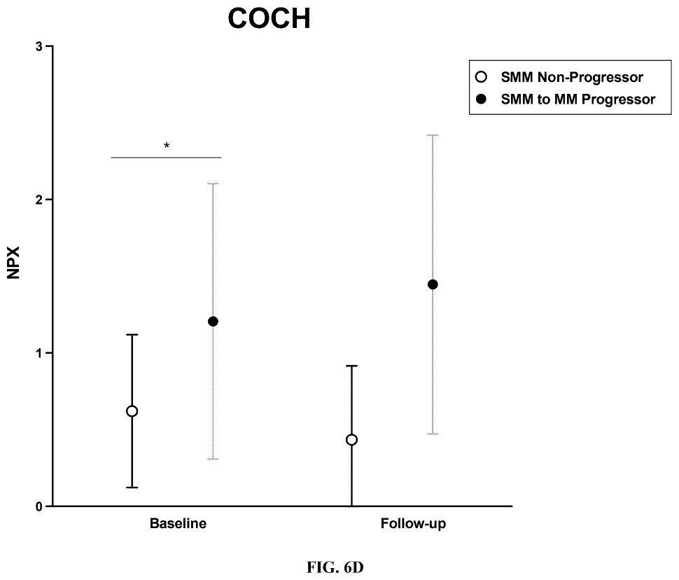

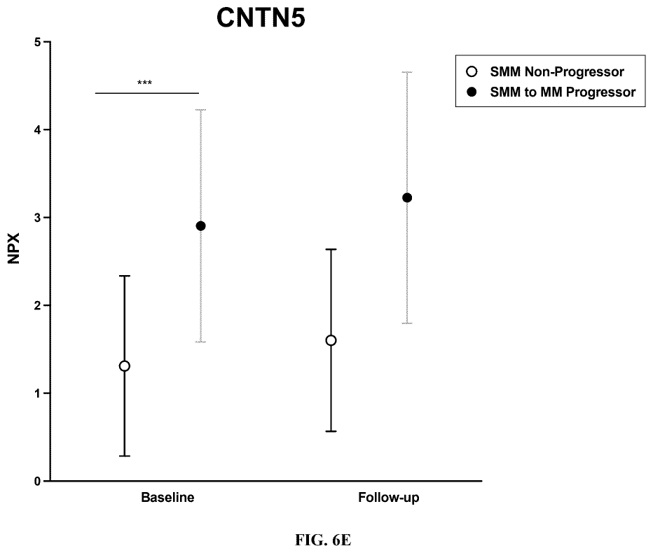

Four proteins that are vital for calcium homeostasis and integrin-mediated cell adhesion were also identified as significantly increased from healthy to MM and were also significantly elevated in SMM progressors vs. SMM non-progressors, indicating that these proteins may be used as biomarkers of high-risk disease (FIG. 6A-6E). In total, these five protein markers (i.e., TNFRSF17/BCMA, TNFRSF13B/TACI, ICAM3, COCH, and CNTN5) were identified as key markers useful for determination of disease progression risk and/or state.

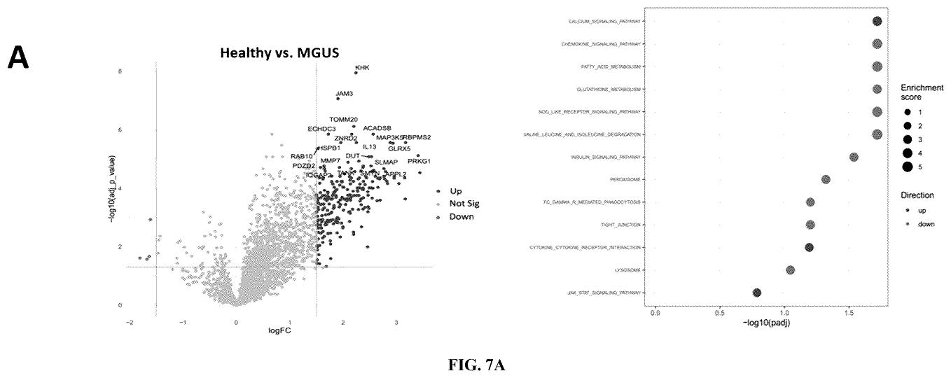

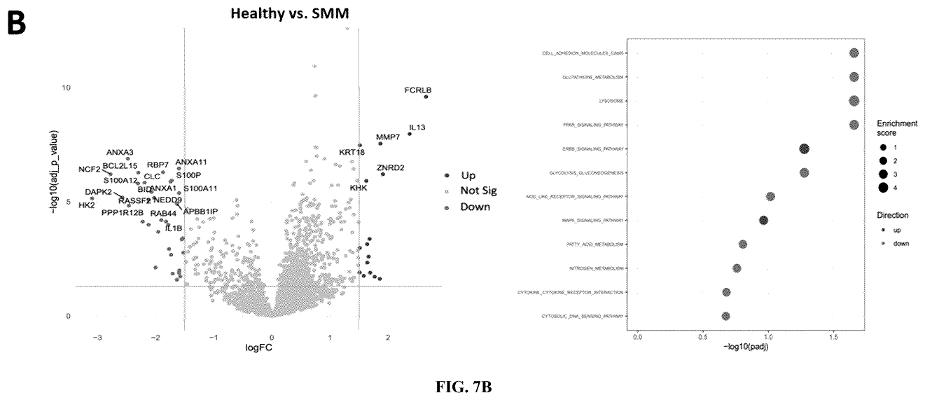

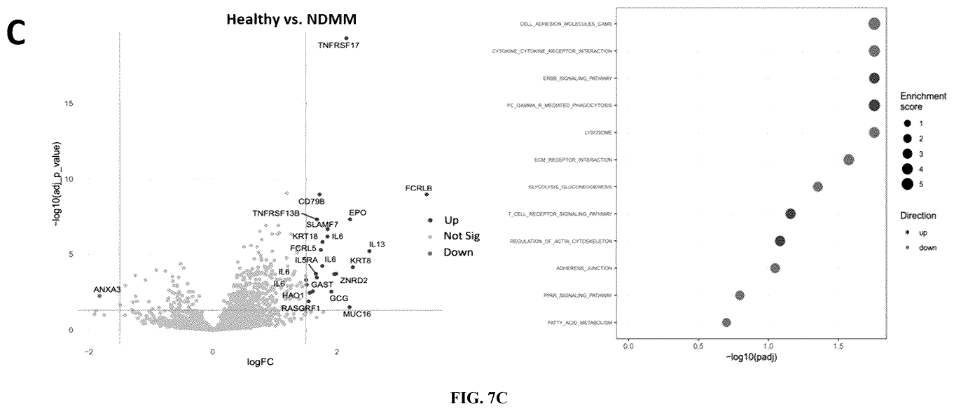

Gene Set Pathway Enrichment analyses were also conducted for each of Healthy vs. MGUS, SMM and NDMM. Many proteins were discovered that were significantly differentially expressed in healthy vs. MGUS patients (FIG. 7A), healthy vs. SMM patients (FIG. 7B), and healthy vs. NDMM patients (FIG. 7C). GSEA analysis also revealed the top 12 pathways dysregulated for each disease stage comparison.

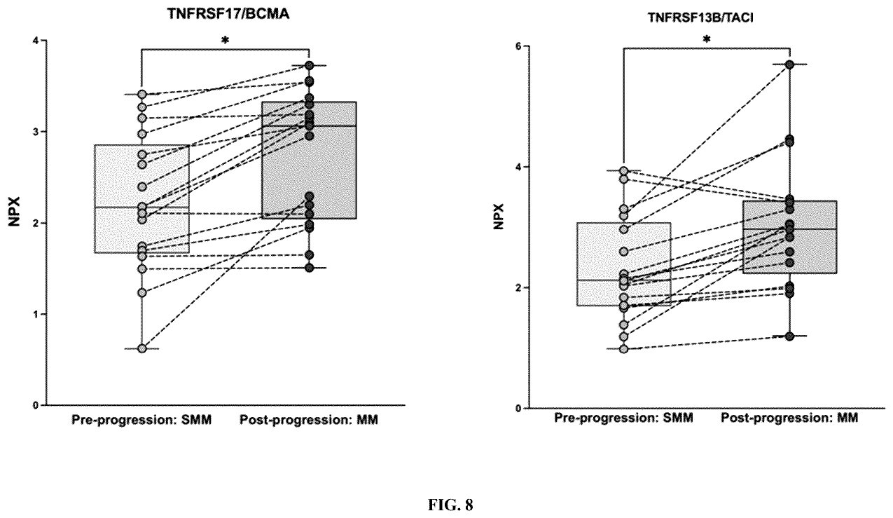

Both SMM timepoint peripheral blood samples and sequential NDMM peripheral blood samples after progression to myeloma disease, but prior to any therapy were taken from 17 patients and examined (FIG. 8). P-values were computed using Wilcoxon's rank-sum test and adjusted using the Benjamini Hochberg approach. Not only were TNFRSF17 and TNFRSF13B plasma levels elevated at the baseline SMM timepoint in progressors vs. non-progressors but there was a significant dynamic increase in expression of these two proteins in sequential samples from SMM-MM progressors. This indicated that not only could the 5 protein markers (i.e., TNFRSF17/BCMA, TNFRSF13B gene/Transmembrane Activator and CAML Interactor (TACI) protein, ICAM3, COCH, and CNTN5) be used in tandem with current clinical markers at the precursor stage to identify patients at high risk of progressing to MM who may benefit from earlier therapy or closer disease surveillance, but gradual increases in TNFRSF17 and TNFRSF13B were additional indicators of disease evolution.

The most comprehensive plasma proteomics study to date was performed herein, which characterized disease stage proteomes and identified candidate high-risk disease biomarkers in longitudinal progressor samples. Five biomarkers for MM disease progression (COCH, ICAM3, TNFRSF13B, TNFRSF17, and CNTN5), including four novel biomarkers (COCH, ICAM3, TNFRSF13B, and CNTN5) were identified, and the results indicate that these biomarkers may be used either alone, or in combination with other biomarkers for MM disease progression such as BCMA, for the detection of MM disease progression in subjects.

### Example 2: scRNA-seq of Paired BM that Underwent Plasma Proteome Profiling

Single-cell RNA sequencing (scRNA-seq) data of both tumor cells and immune microenvironment cells from the bone marrow were integrated to examine expression differences in different cell types.

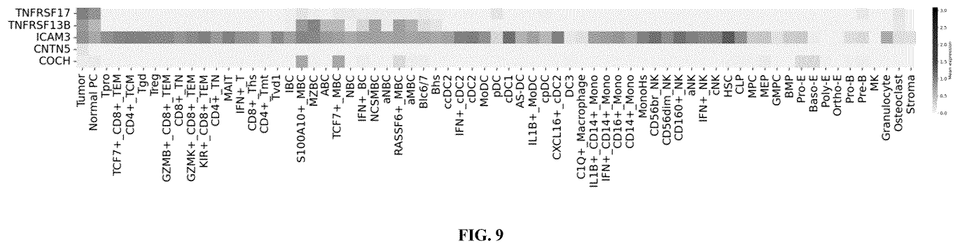

A subset of 86 individuals (13 healthy donors, 14 MGUS, 41 SMM, 18 MM) had single-cell RNA sequencing (scRNA-seq) performed on tumor and immune cells from paired peripheral blood and bone marrow collected at the same timepoint as plasma proteome samples to enable cellular mapping of signals detected in plasma (FIG. 9). Across 69 different BM cell types, it was found that the five-myeloma progression-predictor plasma proteins (COCH, ICAM3, TNFRSF13B, TNFRSF17, and CNTN5) were expressed in plasma cells. Additionally, after malignant plasma cell annotation using V (D) J sequences, TNFRSF17, TNFRSF13B, ICAM3 and CNTN5 were also demonstrated to be upregulated specifically in malignant plasma cells compared to non-malignant plasma cells in patients. This indicated that the progression predictor proteins were a reliable readout of not only plasma cell burden but tumor burden. Meanwhile, TNFRSF13, ICAM3 and COCH were also expressed in various immune cell subtypes, indicating that the interplay of other cell types in the immune microenvironment involved in progression was also reflected in the plasma.

The relationship between the predictor proteins with plasma cell burden holds clinical importance since MGUS and SMM patients may not undergo BM biopsies during routine assessments and strictly rely on few biomarkers present in the peripheral blood, thus, these proteins can act as accessible surrogate biomarkers of BM disease burden. Additionally, MM is a multifocal, spatially heterogeneous disease, where a single-site BM biopsy alone may not provide comprehensive information on tumor burden as well as the underlying disease biology occurring throughout the body. Therefore, this demonstrates that multiomic data can be used to enable cellular mapping of progression-related signals detected in the peripheral blood and identify readouts of plasma tumor burden that can help more accurately stage patients on the MGUS-MM disease spectrum or identify patients who may benefit from earlier treatment intervention.

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

All patents and publications mentioned in this specification are herein incorporated by reference to the same extent as if each independent patent and publication was specifically and individually indicated to be incorporated by reference.

What is claimed is:

1. A panel comprising at least one capture reagent that specifically binds two or more polypeptide markers selected from the group consisting of: B-cell maturation antigen (TNFRSF17/BCMA), cochlin (COCH), contactin 5 (CNTN5), intercellular adhesion molecule 3 (ICAM3), and TNF receptor superfamily member 13b (TNFRSF13B/TACI), or polynucleotides encoding said markers.

2. The panel of claim 1, further comprising the two or more polypeptide markers bound to the at least one capture reagent.

3. The panel of claim 2, wherein the capture reagent is fixed to a solid substrate.

4. The panel of claim 3, wherein the solid substrate is a plate, a chip, a bead, a microfluidic platform, a membrane, a planar microarray, or a suspension array.

5. The panel of claim 1, wherein the at least one capture reagent is an antibody or antigen binding fragment thereof.

6. The panel of claim 1, wherein the at least one capture reagent is an aptamer, or a probe.

7. A method of treating a selected subject having a multiple myeloma precursor disease, the method comprising:
administering an effective amount of an agent for treating a multiple myeloma precursor disease to a selected subject,
wherein the subject is selected by detecting an increase in a polypeptide marker selected from the group consisting of: COCH; ICAM3; TNFRSF13B; and CNTN5, or detecting an increase in a polynucleotide encoding said marker in a biological sample of a subject relative to a reference.

8. The method of claim 7, wherein the detecting is by immunoassay.

9. The method of claim 7, wherein the detecting is by measuring hybridization to a detectable probe or an aptamer.

10. The method of claim 7, wherein the agent comprises one or more of an immunomodulatory agent, a proteasome inhibitor, a corticosteroid, and a CD38 monoclonal antibody.

11. A method for characterizing multiple myeloma precursor disease in a subject, the method comprising:
detecting one or more polypeptide markers selected from Table 1, Table 2, Table 3, or Table 4, or a polynucleotide encoding said one or more polypeptide markers in a biological sample from the subject,
thereby characterizing the multiple myeloma precursor disease in the subject,
wherein the polypeptide marker is not a B-cell maturation antigen (TNFRSF17/BCMA).

12. The method of claim 11, wherein the one or more polypeptide markers is selected from the group consisting of: cochlin (COCH); contactin 5 (CNTN5); intercellular adhesion molecule 3 (ICAM3); and TNF receptor superfamily member 13b (TNFRSF13B).

13. The method of claim 11, wherein the method characterizes the disease as low-risk smoldering multiple myeloma (SMM) or high-risk SMM.

14. The method of claim 13, wherein the one or more polypeptide markers is selected from the group consisting of: TNF receptor superfamily member 13b (TNFRSF13B), contactin 5 (CNTN5), Fc receptor-like protein 5 (FCRL5), TNF receptor superfamily member 13 (TNFSF13), intercellular adhesion molecule 3 (ICAM3), CD5 antigen-like (CD5L), SLAM family member 7 (SLAMF7), cluster of differentiation 79B (CD79B), marginal zone B and B1 cell specific protein (MZB1), interleukin receptor 5 subunit alpha (IL5RA), and neuronal-specific septin-3 (SEPTIN3).

15. A method for monitoring multiple myeloma precursor disease progression in a subject, the method comprising:
a) detecting a polypeptide marker selected from the group consisting of: cochlin (COCH), contactin 5 (CNTN5), intercellular adhesion molecule 3 (ICAM3), and TNF receptor superfamily member 13b (TNFRSF13B) or a polynucleotide encoding said marker in two or more biological samples from the subject, wherein the first sample is collected at an earlier point in time than the second sample; and
b) comparing a level of the markers, wherein an increase in the level is indicative that the multiple myeloma precursor disease has progressed in the subject, and failure to detect an increase in the level is indicative that the multiple myeloma precursor disease has not progressed.

16. The method of claim 15, wherein the subject is undergoing treatment for multiple myeloma precursor disease.

17. The method of claim 7, wherein the biological sample is one or more of: blood, serum, plasma, or bone marrow biopsy.

18. A method for assessing a subject's risk of multiple myeloma precursor disease progression, the method comprising:
detecting a polypeptide marker selected from the group consisting of: cochlin (COCH), contactin 5 (CNTN5), intercellular adhesion molecule 3 (ICAM3), and TNF receptor superfamily member 13b (TNFRSF13B) or a polynucleotide encoding said marker in a biological sample from the subject,
thereby assessing the risk of multiple myeloma precursor disease progression in the subject.

19. The method of claim 18, wherein the assessing the subject's risk of multiple myeloma precursor disease progression comprises assessing the risk that the multiple myeloma precursor disease will, within a timeframe, progress into multiple myeloma.

20. The method of claim 18, wherein the assessing the subject's risk of multiple myeloma precursor disease progression comprises identifying the multiple myeloma precursor disease as a high-risk multiple myeloma precursor disease.

21. The method of claim 20, wherein the biological sample is one or more of: blood, serum, plasma, or bone marrow biopsy.

22. The method of claim 20, wherein the multiple myeloma precursor disease is monoclonal gammopathy of undetermined significance (MGUS) or smoldering multiple myeloma (SMM).

23. The method of claim 7, further comprising detecting a B-cell maturation antigen (TNFRSF17/BCMA), or a polynucleotide encoding BCMA in a biological sample from the subject.
